(19) United States
(12) Patent Application Publication
(10) Pub. No.: US 2014/0147533 A1
(43) Pub. Date: May 29, 2014

(54) FEED SUPPLEMENT PRODUCTS AND METHODS OF USING SUCH PRODUCTS FOR IMPROVED RAISING OF RUMINANT LIVESTOCK ANIMALS

(71) Applicant: TUSKEGEE UNIVERSITY, Tuskegee, AL (US)

(72) Inventors: Sandra SOLAIMAN, Tuskegee, AL (US); Byeng Ryel MIN, Tuskegee, AL (US)

(73) Assignee: TUSKEGEE UNIVERSITY, Tuskegee, AL (US)

(21) Appl. No.: 13/686,520

(22) Filed: Nov. 27, 2012

(51) Int. Cl. A23K 1/18; A61K 36/15

(52) U.S. Cl. CPC A23K 1/1813; A61K 36/15
USPC 424/770; 426/635; 426/2

(57) ABSTRACT

Embodiments of the present invention relate to the use of tannin-containing wood products in animal feed to improve production efficiency and health of ruminants (e.g., sheep, goats, and cattle) by reducing internal parasite load, reducing methane and ammonia production in the rumen, and decreasing phosphor emissions from fecal waste. Embodiments include a domesticated ruminant feed comprising a condensed tannin. Certain embodiments relate to methods comprising administering condensed tannins to ruminant animals by incorporating pine bark or other suitable condensed tannin-containing wood products into regular animal feed.

### FIELD OF THE INVENTION

The present disclosure relates to the use of tannin-containing wood products in animal feed to improve production efficiency and health of ruminants (e.g., sheep, goats, cattle and horses) by reducing internal parasite load, reducing methane and ammonia production in the rumen, and decreasing phosphor emissions from fecal waste. More particularly, the present disclosure relates to methods that comprise administering condensed tannins to ruminant animals by incorporating pine bark or other suitable condensed tannin-containing wood products into regular animal feed.

### BACKGROUND

Gastrointestinal parasitic infections are generally regarded as the most prevalent and important health problems of grazing ruminant livestock animals in the southeastern United States. Most of the economic losses caused by internal parasites are actually not due to mortality, but production loss.

The most common approach for controlling gastro-intestinal parasites in ruminants is the use of anthelmintics. These drugs are costly and eventually worms develop that are resistant to even the most effective drugs in a short time. Some alternatives have been introduced, such as using bioactive plant compounds such tannins. Elevated levels of tannins has been shown to provide antihelmitic effects with respect to the decrease of gastrointestinal parasites in ruminants fed a condensed tannin diet (Min & Hart, 2003). Goats fed a diet of condensed tannins have also shown a decrease in gastrointestinal microorganisms (Lee, Vanguru, Kannan, Moore, Terrill, & Kouakou, 2009). Tannins have also been shown to increase the amount of undegraded protein in ruminants, thereby increasing the availability of usable protein in the ruminant (Puchala, Min, Goetsch, & Sahlu, 2005). Tannins possess the ability to cause toxicity in ruminants, although studies have shown that compared to other ruminants, goats are less affected by compounds such as tannins found in plants (Foley, Iason, & McArthur, 1999; Puchala et al., 2005). Molan, Attwood, Min, & McNabb (2001) reported that tannins extracted from Lotus corniculatus had a detrimental effect on survival of certain rumen microorganisms. Rumen bacteria are responsible for biohydrogenation of dietary poly-unsaturated fatty acids, the products of which include conjugated linoleic acid and saturated fatty acids (Priolo & Vasta, 2007). If biohydrogenation is reduced, the potential for oxidation of poly-unsaturated fatty acids in the tissue may be increased as a result of feeding high levels of dietary tannins. Conversely, derivatives of commercial tannins product added directly to ground beef have been shown to have antioxidant effects and reduce lipid oxidation of cooked patties (Ahn, Grun, & Fernando, 2006).

Most of the studies investigating the effects of using bioactive plant compounds in feed against gastrointestinal nematodes in the United States have focused on forage legume sericea lespedeza or sericea lespedeza pellets. However, this legume must be cultivated with high cost of farmland, planting equipment, processing and handling fees that are not easily applicable and are costly. Thus, the present state of the art for ruminant livestock raising lacks an acceptable approach for introducing bioactive plant compounds into livestock feed to address gastrointestinal parasitic infections.

Globally, livestock are the largest source of methane production from human-related activities. For the United States, livestock is the third largest source of methane production. Livestock production can also result in emissions of nitrous oxide and carbon dioxide and the run-off of phosphorous into the water table. There are various approaches currently used to reduce greenhouse gas emissions from livestock production, which include management strategies that improve production efficiency and result in lower emissions per unit of milk or meat produced. Such current approaches, however, are often costly and difficult to implement.

Goat meat, also known as chevon, is a lean source of high quality protein that is consumed in many parts of the world to differing degrees. Goats are adaptable animals that can be raised in a variety of climates, thereby increasing their popularity as meat animals. While goat meat consumption in the United States is primarily confined to ethnic groups, demand for goat meat in the United States has often outstripped supply and goat meat is a growing sector of the United States meat industry. One of the most popular breeds raised by goat ranchers in the United States is the Kiko, which was developed in New Zealand as a hardy breed that has shown great reproductive success.

With the adaptability of goats to a variety of climates and diets, there is a certain degree of variability in goat diets. This, in turn, can impact the yield and quality of chevon obtained from raised goats. Thus, the use of feed supplements in goats in particular is complicated by the chances that such supplements may negatively impact meat or milk quality.

Processing technologies such as marination have been shown to improve meat quality, thus adding value to meat products. Injection with salt, phosphate, and water generally has been shown to improve consumer acceptance (Detienne, Reynolds, & Wicker, 2003). Further, even though goat meat has gained popularity in the United States, little research has been done to evaluate value-added processing of chevon in particular. Other studies have also shown marination to improve objective meat tenderness, taste, and also increase cook yields in red meats such as pork (Sheard & Tali, 2004) and to improve sensory characteristics of lamb (Sawyer, Brooks, Apple, and Fitch, 2009). Value-added processing of chevon has the potential to help develop more marketable goat meat products to extend consumption in the U.S.

Thus, there is a need in the industry for natural feed supplements for improving ruminant animal feed efficiency, suppressing fecal egg count (a commonly accepted indicator of internal parasites load), and reducing environmental impact of livestock herds in domesticated ruminants without negatively impacting food product quality or restricting down-stream value-added processing of the products.

### SUMMARY OF THE INVENTION

The various embodiments of the present invention relate to the finding that certain commonly-available wood processing by-products may be used to improve production efficiency and health of ruminant feed-stock animals (sheep, goats, cattle and horses). In particular, the present invention relates to the finding that incorporation of a wood product that contains appreciable levels of tannins, and condensed tannins in particular, into the feed of ruminants decreases fecal egg counts, and fecal methane gas production from ruminants through decreasing methanogenesis, while also increasing meat yields without negative impact upon meat quality.

The various methods of the present invention use the tannin-containing wood product as an additive in a total mixed ration to the animal's regular feed to improve animal feed efficiency, and decrease methane gas production and fecal egg counts, in domesticated ruminants. In this regard, the methods of the present invention decrease the risk of metabolic diseases and internal parasites contamination in a cost-effective and sustainable manner.

Further, the various embodiments of the present invention comprise methods that incorporate tannin-containing wood products into a mixed food ration delivered to a ruminant domesticated animal to improve meat yield without negatively impacting consumer-perceived meat quality.

Additionally, the various embodiments of the present invention comprise methods that incorporate tannin-containing wood products into a mixed food ration delivered to a ruminant domesticated animal to reduce the oxidative of meat produced by such animal. As such, methods of the invention may be used to enhance the shelf life of meat produced by the subject animals and produce safer meat. The methods may also be used to reduce oxidative stress of animal tissues, and/or enhance the immune system of the animal, causing the animal to perform better.

Furthermore, the various embodiments of the present invention comprise methods that incorporate tannin-containing wood products into a mixed food ration delivered to a ruminant domesticated animal to improve meat yield without negatively impacting consumer-perceived meat quality.

Also, the present invention comprises methods for reducing methane and ammonia production in the rumen, and thereby reduce and/or eliminate associated bloat. Bloat, which can be related to or the result of a high-grain diet or high legume forage diet (e.g., alfalfa, winter wheat forages) in ruminants is a significant problem in feedlot or grazing livestock production systems.

Additional embodiments of the invention include methods for reducing the amount of phosphorous released from the feces of ruminant animals by administering the ruminant animal a diet that incorporates tannin-containing wood products into a mixed food ration. Tannin is a chelator with the potential to bind minerals and release them slowly over time. As such, increased tannin intake in a ruminant diet in accordance with the present invention may be used to reduce phosphor run-off, which is a significant problem in situations where a large amount of ruminants may be concentrated (e.g., commercial ranching operations, dairy farms, etc.). Thus, preferred methods of the invention utilize tannin-containing wood additives to decrease internal parasites and increase animal gain efficiency while reducing livestock production impact on environment (reduced methane, ammonia, skin pathogens).

Suitable tannin-containing wood products can be obtained from single pine tree species, a mixture of pine species, or from other tree species that contain similar levels of tannins without also containing unwanted toxins or contaminants. However, preferred embodiments of the present invention comprise methods that utilize tannin-containing pine bark, which is readily available and has a long history of production and use as mulch in the timber industry in the southeastern United States. Pine bark is a natural product that is generally regarded as safe (GRAS). Plants that are considered toxic due to hydrolysable tannins, such as harendog (Clidemia hirta), oak (Quercus ilex), yellow wood (Terminalia oblongata), and supple jack (Ventilago viminolis) may not be suitable depending upon the level of toxicity provided by the plant product to the specific ruminant species in question. The various methods of the present invention preferably use pine bark in a total mixed ration in the ruminant animal's regular feed to improve animal feed efficiency, and decrease methane gas production and fecal egg counts, in domesticated ruminants.

Embodiments of the invention include a domesticated ruminant feed comprising a non-toxic tannin-containing wood product, preferably pine bark wood product. In certain embodiments, the feed contains pine bark in the range of 15-30% of the total feed by weight. In other embodiments, the feed contains pine bark in the range of 5-35%, 5-30%, 10-35%, 10-35%, 10-15%, 15-35%, 15-20%, or a range that supplies sufficient quantities (to achieve the desired goals as set forth herein) of condensed tannins and is tolerable to the animal. The tannin-containing wood product such as pine bark preferably comprises condensed tannins.

In certain embodiments, the feed comprises condensed tannins in the range of 0.19% to 3.2% or in the range of 1.63% to 3.2% or 0.19% to 3.2%.

Embodiments of the invention include a method a method of decreasing internal parasites comprising feeding a domesticated ruminant feeds containing pine bark or condensed tannins. The internal parasites are selected from the group consisting of E. coli., Flavobacteriaceae, Acinetobacter, Acinetobacer-baumannii, moraxellaceae. Preferably the fecal egg counts are reduced by at least 50%. In certain embodiments the resistant worms (internal parasites) are eliminated.

Embodiments of the invention also include a method of decreasing fecal methane gas emissions by decreasing methanogenesis in domesticated ruminants by feeding feeds containing pine bark or condensed tannins.

Embodiments of the invention also include a method of increasing feed efficiency by altering ruminal fermentation in domesticated ruminants by feeding feeds containing pine bark or condensed tannins.

Embodiments of the invention also include a method of reducing the amount of phosphorous released from feces of ruminant animals in domesticated ruminants by feeding feeds containing pine bark or condensed tannins.

While the experimental methods described below utilize loose mixtures of ground pine bark with grain as feed, one skilled in the art will understand that other feed forms are permissible. Preferably, the various embodiments of the present invention comprise a self-fed supplement (e.g. pellet or other forms), and methods of using such supplements, that will deliver efficacious dose levels of the tannin by incorporating a suitable amount of tannin-containing wood additive mixed with grains or grasses that are commonly used for ruminant feed.

The following description of experiments and data below with reference to the various tables and drawings is intended to depict only typical embodiments of the invention and do not therefore limit its scope.

### BRIEF DESCRIPTION OF THE FIGURES

FIGS. 1A and 1B show the results of a study showing a drop in pH (FIG. 1a) and temperature of goats longissimus muscle (LM) (FIG. 1b) during 24 hours.

### EXPERIMENTS AND DATA SUPPORTING THE INVENTION

The various experiments described hereafter illustrate the use of pine bark as a natural anthelmintic and anti-microbial for internal parasite mitigation and food safety, and as feed additive to improve production efficiency while reducing livestock production impact on environment through reduction of fecal methane gas emissions and ammonia production, supporting its use as feed additive in the animal livestock industry.

### Experiment 1

Eighteen Kiko-cross goats (33.4±0.98 kg; n=6) were used to determine the impact of pine bark (PB), which contains condensed tannins (CT), such as Proanthocyanidins), on dry matter (DM) intake, fecal dry matter output, fecal bacterial diversity and in vitro methane gas production. PB supplementation to a base wheat straw (WS) and standard grain mix diet occurred as follows, with 7 days total fecal collection and 2 treatment periods. The 18 subject goats were assigned to one of three experimental treatment regimens that included: the control diet of 0% PB and 30% WS (0.17% CT DM); 15% PB and 15% WS (1.6% CT DM) and 30% PB and 0% WS (3.2% CT DM) as fed. Freshly dried PB and WS were finely (1.5-3 mm) ground and incorporated in the grain mix portion of the diet to provide 0 g, 16 g, and 32 g CT/kg DM in 0%, 15%, and 30% PB diets. Fecal bacterial populations were measured using a 16S-based pyrosequencing technique to characterize and elucidate changes in bacterial diversity among the diets. Fecal samples were collected from each goat for sequencing analysis. In vitro methane gas production was measured as plunger displacement (cc) at 0 to 24 hour incubation periods with fecal inoculants that were obtained from goats in the three diet classes. Total methane gas production was estimated from total DM fecal output and in vitro methane gas production per unit of fecal material. An average fecal DM output was linearly increased with increased PB supplementation (375:386:460 g DM/animal; P<0.04), but estimated methane gas (291, 158, and 51 cc/day/goat; P<0.01) and in vitro methane (0.77, 0.42, and 0.11 cc/g of feces) gas production (P<0.001) decreased (linearly) as the level of PB supplement increased (0, 15, and 30% PB) in the diet, respectively.

Predominant fecal genera were Flavobacteriaceae (up to 18%), Oscillibacter (up to 15%), and Oscillibacter spp. (up to 17%) microbial population in control (0% PB), 15 and 30% PB, respectively. The proportion of Flavobacteriaceae (25, 4.5, and 3%), Acinetobacter (4.6, 3.1, 4.1%), Acinetobacter-baumannii (4.9, 3.0, and 5.8%), Moraxellaceae (4.4, 1.1, 1.2%), and E. coli (6.3, 2.1, and 2.1%) population decreased as the level of PB supplement increased in the diet, respectively. Archaea population varied among diets (1.03, 0.56, and 1.15%, respectively).

These results to Experiment 1 indicated that feeding PB reduced methane gas and E. coli population and modified fecal bacterial population.

### Experiment 2

A series of in vivo and in vitro trials utilizing ground pine bark additive over a range of dosages (0, 15 and 30% of total feed intake) with growing Kiko-cross goat kids were conducted under Experiment 2. Goats were strategically de-wormed with commonly used anthelmintic to reduce or eliminate gastro-intestinal parasites; however, most resistant worms survived under controlled environment. The most significant finding of this work was that average fecal egg counts (an indication of parasite load) was reduced by 52 to 56% with 15-30% pine bark inclusion (Table 8). More significantly, these were resistant worms that were eliminated. Feeding pine bark at 15-30% of diet improved average daily gain (by 49%) and feed efficiency linearly (Table 2). There was no difference in initial body weight of goats; however, final body weight (9%), cold carcass weight (10.5%), and sirloin (15.3%) yields were linearly increased with increasing pine bark additive (P<0.06-0.01) in the diet (Table 3). Growth of other organs was similar except for liver and hide that were higher as a % of body weight (Table 4). Feeding pine bark was associated with higher feed intake, improved feed efficiency (gain: feed ratio) and enhanced rumen fermentation (low acetate:propionate ratio and lower ammonia level) in pine park supplemented group compared with control (non-pine bark supplemented) (Table 5). In vitro fecal incubation results indicated that feeding pine bark reduced total fecal gas (62%) and methane gas (86%) emission linearly (P<0.001). Similarly, feeding pine bark at low (15%) and high levels (30%) lowered total methane gas production by 45.6 and 82.3%, in vivo, respectively, when accounted for total fecal output (Table 6).

Feeding pine bark also reduced in vitro growth of bacteria on the skin swab samples by 21 to 29% a positive indication of its use as pre- and post-harvest food pathogen mitigation strategies (Table 7). The present study confirmed that ground pine bark additive could affect animal weight gain, carcass yield, rumen fermentation, internal parasites and fecal methane gas emission and skin bacterial population in goats. Therefore, tannins-containing pine bark as a feed additive has the potential to decrease internal parasites and fecal methane gas production, and improve animal performance and feed efficiency by altering ruminal fermentation (volatile fatty acids (VFA) and ammonia productions).

### Experiment 3

For Experiment 3, twenty-two Kiko-cross goat kids (Capra hircus; Body weight, 27.5±1.04 kg) were purchased and were used to quantify the animal performance, rumen fermentation, and carcass traits as affected by PB supplementation. Goat kids, approximately 5 months of age, were stratified by body weight and randomly assigned to the experimental treatment groups in a completely randomized design experiment. Goats were individually housed indoors in pens of approximately 1.2 m² with elevated floors. Animals were examined and drenched with anthelmintic (Cydectin; Moxidectin, Fort Dodge Animal Health, Fort Dodge, Iowa, USA) under supervision of a veterinarian before the experiment commenced. Animals were fed grain mixes containing different levels of PB, and bermudagrass hay (BGH; Cynodon doctylon) at 85:15, respectively. An adjustment period of 4 weeks allowed goats to be acclimated to pen living, routine feeding and to allot time for proper diet adjustment before the start of the study.

The grain mix portion of the different diets contained different levels of the CT-containing ground PB replacing ground wheat straw (WS; Triticum aestivum). Differing diets for this experiment included: the control diet—0% PB plus 30% WS, 15% PB plus 15% WS, and 30% PB plus 0% WS as fed. The fresh PB was donated by a wood processing company, and air-dried under the shed before processing. Freshly dried PB and WS were finely (1.5-3 mm) ground and incorporated in the grain mix portion of the diets to provide 1.9, 16.3, and 32 g condensed tannins (CT)/kg DM in 0, 15, and 30% PB/WS diets, respectively (Table 1). Grain mixes containing ground PB/WS were commercially mixed and were offered daily at 85% of the total ration to each goat, with remaining 15% consisting of Bermuda grass hay.

Animals were fed once a day at 9:00 am and feed offered and refused was monitored for 83 days of growth performance and gain efficiency measurements. Animals had access to water and trace mineral salt block ad libitum. Grain mixes and hay were offered separately and refusals were recorded daily. Amounts of feed offered were adjusted every 3 to 4 days to maintain the preferred daily refusal rate of 5 to 10%.

Diet samples were collected every 2 weeks. Composite samples for grain mixes and ingredient samples for Bermudagrass hay, PB, and WS (n=3) were dried for 48 hours at 55° C. in a convection oven. Samples were then ground in a Thomas-Willey mill to pass through a 1-mm mesh screen. Ground composite samples were analyzed for DM, lignin, non-fiber carbohydrate, ether extract, total digestible nutrient (TDN), minerals and crude protein (CP) according to the methods described by AOAC (1998). Nitrogen (N) was determined using a Kjeldahl N, and crude protein was calculated by multiplying N by 6.25. Dietary neutral detergent fiber (NDF) and acid detergent fiber (ADF) were determined on composite samples according to Van Soest et al. (1991) using an Ankom 200 fiber analyzer and ANKOM F57 filter bags (Ankom Technology Corp., Fairport, N.Y.). Acetone (70%) extractable CT in grain mixes was determined using a butanol-HCL colorimetric procedure (Terill et al., 1992).

Rumen fluid samples were collected (10 mL) at days 0, 40, and 83, via stomach tube, approximately 2 hours after morning feeding. First, 10 mL of the samples were discarded to eliminate contamination with saliva. These samples were treated immediately using 1 mL of 50% (vol/vol) HCl for ammonia-N analysis and with 1 mL of a mixture of metaphosphoric acid (187.5 g/L) and formic acid (250 mL 100%/L) for volatile fatty acids (VFA) samples (Min et al., 1998). These samples were then centrifuged at 17,000 g for 15 min and the treated supernatant was stored at −20° C. for later analysis. Rumen fluid samples were analyzed for volatile fatty acids (Goetsch and Galyean, 1983) and ammonia-N (Chaney and Marbach, 1962).

Blood samples were also collected on days 0, 29 and 72, via jugular vein, in ethylenediaminetetraacetic acid (EDTA) and non EDTA containing vacutainer tubes (Franklin Lakes, N.J., USA) and were analyzed for complete blood counts and blood serum metabolites immediately. Total white blood cell numbers were determined by the method of Natt and Herrick (1952). Lymphocyte population was evaluated from stained blood smears with brilliant crestal blue (Mukkur and Bradley, 1974).

Final body weight was obtained after 83 days, and goats were transported approximately 300 km, kept overnight, and harvested according to USDA guidelines after a 24 hour hold period. Goats were given free access to water during the hold period. On the next morning before harvest, goats were weighed after a 24 hour period without feed and were harvested according to the USDA approved guidelines (USDA, 2001). Transportation shrink (%) was measured by weighing animals before and after transport (overnight fasting live weight). Blood was collected and weighed. The esophagus was ligated, and the head, hooves, and skin were removed and weighted. The rectum was ligated, and the entire alimentary tract was removed and weighted. Non-carcass or internal fat was the sum of visceral and perirenal depots. The carcass and non-carcass organs and tissues were expressed in kg and relative to empty body weight, which was the sum of these components minus digesta mass of the gastrointestinal tract. Post-mortem necropsy examination and dissecting the kidney and liver were according to the method of Nietfeld (2010).

Hot carcass weight was determined on the day of harvest and carcasses were chilled at 4° C. for 24 h and cold carcass weight, and carcass shrink weight were measured. Carcasses were ribbed between the 12th and 13th rib for further evaluation. Fat depth over the midpoint of longissimus muscle (LM) at the 12th rib, body wall fat measured at lower point of the 12th rib, kidney and pelvic fat weight (KPF), dressing percentage (DP), longissimus muscle area (LMA), leg circumference, sirloin, loin, shoulder, breast, and trim weight were determined by trained personnel 24 hours postmortem.

Longissimus muscle pH and temperature were measured at the 12th to 13th rib 1, 3, 5, 7, 12, and 24 hours postmortem using a pH and temperature meter with piercing electrode and temperature probes. Ribbed carcasses were allowed to bloom for approximately 30 minutes at 4° C. and evaluated for objective color measurements.

Instrumental color measurements were taken the 12th rib longissimus muscle area (LMA) with a chromameter and two measurements were taken and averaged to obtain a representative measure of initial lean color. The chromameter was calibrated by using a standard white calibration plate. Color was expressed in terms of Commission Internationale de l'Eclairage values for lightness (L*), redness (a*) and yellowness (b*).

Data obtained from Experiment 3 were analyzed by the Mixed Model procedure of the Statistical Analysis System (SAS, Inst., Inc., Cary, N.C.) for completely randomized design with the factors examined being three levels of PB supplementation in the diets. Linear and quadratic effects were determined utilizing poly-nominal orthogonal contrasts for equally spaced treatments. Animals were the experimental unit and were treated as a random effect. The variables included were diet-composition, feed intake, average daily gain, carcass and non-carcass traits, and blood parameters. Mean separation was performed using Fisher's Protected Least Significant Differences at probability level of P<0.05. Animal body weight change, rumen fermentation parameters, muscle pH and temperature were analyzed as repeated measures with treatment, period, and treatment x period interactions. There was no treatment x period interactions (P>0.10) hence only the main effects are reported for blood parameters in the result section. Data are presented as LS mean values together with the standard error of the mean.

The principal objectives of Experiment 3 were to measure the effects of condensed tannin (CT)-containing ground PB supplementation as a feed additive on gain efficiency, growth performance, rumen fermentation, blood parameters, and carcass characteristics of meat goats. The most significant findings of this study were increased dry matter intake and average daily gain with no detrimental effects on health when goats received CT-containing PB. Addition of PB to the diets improved gain efficiency (grain:feed ratio) partially due to increased intake and changes in rumen fermentation efficiency by decreasing acetate:propionate ratios. Decreased ruminal ammonia may have altered Nitrogen metabolism in the rumen.

Ingredients and chemical composition of experimental grain mixes, PB, WS and BGH are presented in Table 9. Goats were provided diets that met all animals' requirements for growth and gain according to National Research Council (NRC) (2007). Total CT concentration in the PB and WS was 11.1 and 0.03% DM, respectively. However, grain mixes analysis resulted in 0.19, 1.63 and 3.2% CT on % DM for the 0, 15 and 30% PB diets. All the experimental treatments provided similar nutrients, except CT and lignin that was higher in 15 and 30% PB ration.

Body weight, dry matter intake, and growth performance of Kiko-cross goats are summarized in Table 10. Total dry matter intake (linear; P=0.001) and intake of grain mixes (linear; P=0.001) were increased as PB increased in the diets. Similarly, Solaiman et al. (2010) reported that total dry matter intake of growing goats increased as Sericea lespedeza ground hay (6.5% CT in DM) replaced alfalfa meal in the grain mixes and Turner et al. (2005) reported that goats receiving the CT-containing Sericea lespedeza (Lespedeza cuneata) hay (23.1 mg CT/mg soluble protein) had higher dry matter intake than those fed the alfalfa hay based diet. Puchala et al. (2005) also reported increased dry matter intake in Angora does fed CT-containing Sericea lespedeza (17% CT in DM) compared with a mixture of crabgrass (Digitaria ischaemum) and tall fescue (Festuca arundiacea). This may be attributed to the fact that goats naturally prefer browse that contains bioactive plant tannins and alkaloids. Ruminant normally consuming tannin-rich feeds appears to develop defensive mechanisms against tannins (Makkar, 2003). Consequently, browsing animals such as goats, deer and antelopes carry tannins tolerant bacteria (e.g. Streptococcus caprinus; diplococcoid bacterium) and produce tannin-binding salivary protein to overcome negative impact on digestibility in the rumen, whereas this mechanism may be less developed in other species, such as sheep and cattle (Brooker et al., 1994; Nelson et al., 1995; McSweeney et al., 2001). Thus, the amount of PB incorporated into a particular diet may optimally be varied depending upon the particular ruminant species.

There was no difference in initial body weight of goats among treatments; however, final body weight (P=0.06), and average daily gain (P=0.001) improved (linear) as the level of ground PB increased in the diet. The presence of optimum levels of CT in the diet can reduce protein degradation in the rumen and improve by-pass protein flow to the small intestine (Min et al., 2003), thus, enabling more enzymatic hydrolysis of dietary protein in the lower tract (Jones and Mangan, 1977). Min et al. (2003) reported that beneficial effects of CT in the diet on sheep performance may occur in the range of 2 to 4% CT of diet DM. This may partially explain why the growth performance of goats in Experiment 3 was improved for those goats receiving diets containing 15 (1.63% CT, DM) and 30% (3.2% CT, DM) PB compared to control diet (0.19% CT, DM). This has been confirmed by the findings of Solaiman et al. (2010) that average daily gain was improved as tannin-containing Sericea lespedeza increased in the diet up to 30% of total diet (2.22% CT). Although dry matter intake of goats receiving PB diets was increased in the present study, gain efficiency was also improved (linear; P=0.04). This may partially due to the shift in rumen fermentation pattern and lower acetate/propionate ratio in goats fed PB diets reflected in more efficient use of energy in these goats.

Rumen fermentation parameters are summarized in Table 11. There was no effect (P>0.10) of PB supplementation on ruminal ammonia level on days 0 and 40 of the study; however, rumen ammonia was reduced (linear; P=0.003) on day 83. Added PB decreased molar proportion of acetate, and acetate:propionate ratios on day 40 (linear; P=0.01 and 0.001, respectively) and day 83 (linear; P=0.07 and P=0.01, respectively). Molar proportion of propionate and butyrate were varied between sampling times. Beauchemin et al. (2007) reported that supplementation with Quebracho C T (10 or 20 g/kg DM) in growing cattle decreased the molar proportion of acetate, acetate:propionate ratios, and ruminal ammonia compared to control group, not supplemented with tannins. Results from the current study and previous research (Wang et al., 1996) demonstrate that CT consistently decreased the ruminal ammonia and acetate:propionate ratio. One explanation for high average daily gain and gain efficiency in PB supplemented groups in the present experiment may be related to reduced acetate:propionate ratios and increased efficiency of energy utilization; and/or improved protein bypass and utilization, and lowered ruminal ammonia. Higher acetate:propionate ratios are associated with lower average daily gain (Waghorn and Barry, 1987).

Satter and Slyter (1974) reported that rumen ammonia nitrogen concentration below 50 mg/L, as would be found with animals fed a straw diet, limited the synthesis of microbial protein. In the present experiments, diets contained at least 15% CP and rumen ammonia nitrogen concentration was between 82 and 125 mg/liter (Table 3). These levels were not likely to limit rumen microbial protein synthesis. Rumen digestion of carbohydrate is competent in such diets, but by-pass protein was only 65% of N consumed in sheep (MacRae and Ulyatt, 1974) due to excessive degradation of forage protein to ammonia by rumen microorganisms. In the present study, goats consuming CT-containing PB diet had lower rumen ammonia concentration indicating that CT in PB reduced rumen ammonia concentration and improved efficiency of N metabolism in the rumen.

Carcass characteristics of goats fed experimental diets are presented in Table 12. There was no difference in hot carcass weight (HCW), transport shrink, dressing percentage, 12th rib fat thickness, longissimus muscle area (LMA), body wall fat (BWF), leg circle, loin, and kidney pelvic fat (KPF), whereas, cold carcass weight (CCW) (linear; P=0.06), breast, sirloin, and trim traits increased (linear, P=0.01) with addition of PB. The effect of tannins on small ruminant growth depends on the degree of tannins activity. Previous research reported that lambs grazing on high CT-containing forage sulla (Hedysarum coronarium; 5 to 8% CT in DM; Hoskin et al., 1999) had higher cold carcass weight, breast, and sirloin weights compared to those grazing on alfalfa (Medicargo sativa; Niezen et al., 1995). Compared to lambs grazing alfalfa (0.1% CT in DM), lambs grazing CT-containing lotus corniculatus (Birdsfoot trefoil; 3.4% CT in DM) had higher average daily gain, carcass weight, dressing out percentage, and wool growth (Wang et al., 1996). Similarly, Ramirez-Restrepo et al. (2005) reported increase in cold carcass weight, breast, and sirloin weights similar to our study when for lambs fed CT-containing lotus corniculatus.

Organ weights as a proportion of empty body weight are presented in Table 13. Pine bark supplementation had no effect on the organ mass as a proportion of empty body weight for the blood weight, feet, heart and lungs; however, liver and hide weight (linear; P=0.01, and 0.02, respectively) increased (linear) as the level of PB increased in the diet. Gastrointestinal tract weight tended to decrease (linear, P=0.08) in goats fed 15 and 30% PB diets. Feeding CT-containing PB to growing goats in the current study increased liver weight by 15% and hide weight by 16%, and slightly reduced (10%) gastrointestinal tract weight, when compared with control diet. There were no apparent pathological observations on liver or kidney tissues upon examination of these organs.

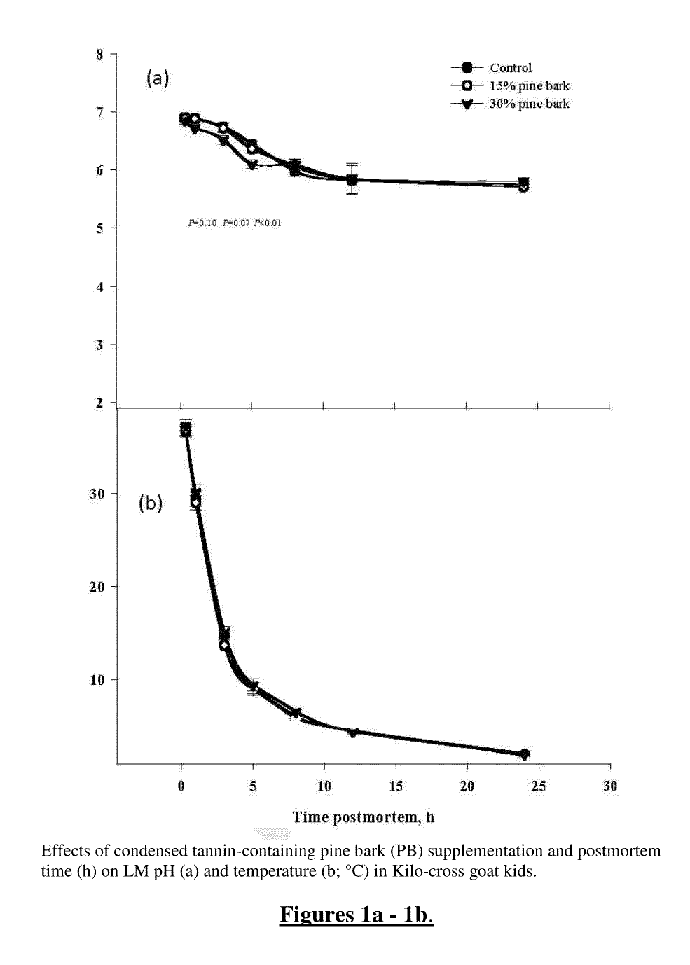

Drop in pH and temperature of goats longissimus muscle (LM) during 24 hours postmortem are presented in FIG. 1a, and FIG. 1b, respectively. There was no difference in muscle temperature drop among treatments; however, goats receiving 30% PB diet had lower (P<0.01) pH within first 10 hours postmortem. The pH was similar among groups between hours of 10 to 24 postmortem. The ultimate pH is important to the chilled meat because it affects its shelf life, color, and quality. High ultimate pH values for goat muscle have been reported in the literature reviewed by Webb et al. (2005). However, goats receiving 30% PB had faster decrease in muscle pH within 10 hours postmortem than other treatments. This may result in carcasses less prone to bacterial contamination and consequently longer shelf life (Warris et al., 1984; Warner et al., 1998). In addition, high ultimate pH has been associated with both malnutrition in ruminants and with long term stress in general, and such meat is normally darker in color than meat with a normal pH (Priolo et al., 2000).

Instrumental color measurements L*, a*, and b* for LM of Kiko cross goats are reported in Table 14. The mechanism of action of tannins on meat color is not clear. Pine bark supplementation in the present study had no effect on the meat color (L*, a*, b*). This is in contrast with finding of Priolo et al. (2000) who reported that lambs fed the CT-containing diets (2.5% CT DM) in carob pulp had a lighter color (higher L*) of longissimus muscle area (LMA) with lower blood hemoglobin concentrations compared to non-CT containing diet. No differences in the lightness of longissimus muscle area (LMA) and blood hemoglobin levels were observed in the present study. The CT-containing PB diets in the present study did not affect redness, yellowness and lightness of meat. However, goats, being a browsing ruminant animal, may utilize CT differently than sheep and cattle and studies comparing CT and non CT-containing forages/browse on meat quality and color, are scarce in goats.

Hemogram and blood serum chemistry of goats consuming different levels of PB are presented in Tables 15 and 16, respectively. These parameters were used as diagnostic tool for screening animal health problems and abnormality. Serum levels of alanine transaminase, aspartate aminotransferase, gamma glutamyltranspeptidase, alkaline phosphatase, and cholesterol are conventionally used for diagnosing human and domestic animal hepatic damage (Silanikove and Tiomkin, 1992). Gamma glutamyltranspeptidase has proven to be a sensitive indicator of minor bovine hepatic damage; alkaline phosphatase and cholesterol are used to detect bile obstruction and mild damage of liver (Silanikove and Tiomkin, 1992). In the present study, there was no difference (P>0.10) in blood serum metabolites and hemogram of goats, except for alanine transaminase, aspartate aminotransferase, albumin, sodium, and chlorine, which decreased (linear; P<0.03) as the level of PB increased in the diet; however, all values fell within the normal range for goats, suggesting that no damage to the liver occurred. This has been confirmed by findings that post-mortem necropsy and dissecting test in this study (no data shown in text) indicated no anatomical lesions on liver and kidney organs. Thus, it appears that goats used in Experiment 3 were well adapted to the PB supplementation up to 30% without suffering any ill effects.

The data from Experiment 3 highlight that CT-containing pine bark has the potential to increase average daily gain and carcass traits by improving gain efficiency and favorable rumen fermentation, with no adverse effect on animal health. Reduction in acetate/propionate ratios supports an improvement in rumen energy efficiency. Pine bark containing CT also lowered ammonia production in the rumen, thereby supporting improvements in protein metabolism.

### Experiment 4

The objectives of Experiment 4 were to evaluate the effects of dietary pine bark (PB) containing condensed tannins (CT) and post butchering enhancement (i.e., salt and phosphate treatment) on processing yield, shelf-life, cooking loss, Warner-Bratzler shear force (WBSF), thiobarbituric acid reactive substances (TBARS), and consumer acceptability of goat loin meat.

Kiko cross goat wethers (n=22) were obtained at approximately five months of age and BW of 27.5±1.04 kg. Animals were examined and drenched with anthelmintic under supervision of a veterinarian before the experiment commenced. Goats were housed and fed individually in pens of approximately 1.2 m² with elevated floors, and given an adjustment period of 4 weeks prior to the start of the feeding trial. Animals were randomly assigned to one of 3 dietary treatments: 0% PB, 15% PB, and 30% PB. Diets contained different levels of the CT-containing ground PB replacing ground wheat straw (WS) as follows: 0% PB-0% PB plus 30% WS, 15 PB-15% PB plus 15% WS, and 30 PB-30% PB plus 0% WS as fed. Fresh PB was air-dried prior to processing. Freshly dried PB and WS were finely (1.5-3 mm) ground and incorporated in the grain mix portion of the diets to provide 1.9, 16.3, and 32 g CT/kg DM in 0, 15, and 30% PB/WS diets, respectively (Table 17). Grain mixes containing ground PB/WS were commercially mixed at the local feed mill and were offered daily at 85% of the total ration, with remaining 15% consisting of Bermuda grass hay. There were 8 goats assigned to the OPB group, and 7 goats each assigned to the 15 PB and 30 PB groups. Animals had access to water and trace mineral salt block ad libitum. Amounts of feed offered were adjusted every 3 to 4 days to maintain the preferred daily refusal rate of 5 to 10%.

On the day prior to harvest, animals were transported and then held overnight with access to water but not feed. Animals were humanely harvested, and carcasses were chilled overnight in a 2° C. cooler. At 24 hour post mortem, carcasses were fabricated, and whole loins [Institutional Meat Purchase Specifications (IMPS) 11-X-50)); goat loin] were collected from both sides of each carcass. Loins were paired by carcass, vacuum packaged, and frozen at −20° C. for 4 months.

Loins thereafter were thawed overnight prior to the start of processing, then boned out to fabricate a boneless loin from each carcass side. Each boneless loin was weighed and individually identified. The left loins from each carcass were assigned to the Non-enhanced treatment (N), and the right loins were assigned to the Enhanced treatment (E). Brines were formulated to a targeted pickup of 20% and final concentrations of 0.5% salt and 0.5% sodium tripolyphosphate for each individual loin. Boneless loins were placed in individual bags with the calculated amount of brine. Bags were tied, placed in a vacuum tumbler and tumbled at 20 RPMS for 20 min at 137 kPa pressure. After tumbling, loins were removed from bags and weighed to determine pickup.

To evaluate shelf life, two 1.5 cm chops were cut from the anterior portion of each loin (both E and N treatments), placed in individual Styrofoam trays, and overwrapped. Trays were placed in a simulated refrigerated display case at 2° C. under cool white lights. The processing day included boning out, marinating (E treatment only) and cutting chops, and was defined as day 0. On day 1, day 3, and day 5, objective color (L*, a*, b*) was evaluated using a Konica Minolta Chroma Meter. The same chop was used to evaluate color on each day.

On day 1 and day 5, thiobarbituric acid reactive substances (TBARS) were evaluated using a modification of the procedure described by Rojas & Brewer (2007). Chops were trimmed of external fat and connective tissue, then homogenized using an Oster 3 Cup Food Chopper. Duplicate 5 g samples of tissue were weighed, then blended for 30 seconds with 1 mL of 0.2 mg/mL BHT and 45.5 mL of 10% trichloroacetic acid in 0.2 M phosphoric acid in a Waring blender. The homogenate was filtered through Whatman no. 1 filter paper, and filtrate was collected in a flask. Two 5 mL aliquots of filtrate were collected from each flask and transferred into glass test tubes. For each sample, 5 mL of 0.02 M thiobarbituric acid (TBA) was added to one tube, and 5 mL deionized water was added to the other tube to be used as a blank for that sample. A standard curve (0, 1.25, 2.5, 5.0, and 7.5 mg malondialdehyde (MDA)/mL) was set up using 25 μM TEP, 0.2 M TBA, and 10% trichloroacetic acid in 0.2 M phosphoric acid. All tubes were capped, inverted to mix, and stored in a dark cabinet at room temperature for about 18 hours. Samples, blanks, and standards were read at 532 nm using a Shimadzu UV-VIS Spectrophotometer. Sample readings were compared against the standard curve and corrected for dilutions to report TBARS values as mg malondialdehyde (MDA) per kg tissue.

The remaining loin sections that were not used for shelf life were individually identified, vacuum packaged, and frozen at −20° C. for subsequent consumer evaluation. Consumer testing took place on 2 days, with approximately half the loins used on each day. Loins were thawed overnight prior to consumer evaluation, removed from vacuum packages, and weighed prior to cooking. Loins were placed on metal baking sheets and roasted in a 177° C. oven to an internal temperature of 71° C., as monitored by a digital thermometer. Loins were weighed after cooking to determine cook loss. After cooking, a 2-cm portion was cut from the anterior end of each loin, and reserved for Warner-Bratzler shear force (WBSF) analysis. The remaining sections were cut into approximately 1-cm cubes and served to consumer panelists. Consumers (n=60 on the first day and n=51 on the second day) were served 6 samples, one representing each treatment combination of dietary pine bark and enhancement. Each loin was identified by a random 3-digit number. Panelists were asked to evaluate appearance, aroma, texture, flavor, and overall acceptability on a 9-point hedonic scale where 1=dislike extremely and 9=like extremely. Two follow-up yes/no questions were included at the end of the evaluation form, including “Have you ever consumed chevon (goat meat) before?” and “Would you consider purchasing a goat meat product similar to the ones tasted if it was available in the supermarket?” Panelists were seated in individual booths under red lighting and provided with water, apple juice, and unsalted crackers to cleanse the palate.

After the completion of consumer testing, the reserved loin sections were evaluated for WBSF. Samples were cooled at room temperature for 3 hours or until internal temperature reached about 25° C. Two 1.3-cm cores were removed from each sample parallel to the muscle fiber orientation. Cores were sheared using an Instron Universal Testing System with a Warner-Bratzler shear force attachment and a 500 N load cell and a crosshead speed of 200 mm/min. Peak force of each core was measured and a mean peak force was determined for each sample. The experimental design thus was a 2×3 factorial in a completely randomized design, with 2 enhancement treatments (E and N) and 3 pine bark treatments (0, 15, and 30% of diet). The experimental unit was individual loin, and the independent variables were enhancement treatment and pine bark level. The dependent variables included pump yield, cooking loss, TBARS values, color values, WBSF, and consumer acceptability scores.

The Univariate procedure of SAS Version 9.2 was used to test for normality of data distribution and to calculate means. Data were analyzed using the Mixed procedure of SAS Version 9.2 to evaluate data for main effects and interactions of dietary pine bark and enhancement. For objective color evaluation, the repeated statement was used with the autoregressive covariance structure to account for the fact that the same chop was used to evaluate color on each day. Analysis of both objective color and TBARS values included the effects of bark, enhancement, and day of storage. For WBSF and consumer evaluation data, the effect of day of testing was not significant (P>0.05) and was dropped from the model. For all tests, significance was determined at an alpha level of 0.05; when significant differences were detected, means were separated using the “lsmeans” statement with the “pdiff” option.

Marinade pickup was calculated using the formula: [Pickup=((Enhanced weight−Initial weight)/Initial weight)*100]. Dietary PB did not affect marinade pickup (P=0.4459; data not shown). Mean pickup of all boneless loins was 12.45% with a standard error of 0.49%. This was lower than the targeted pickup of 20%, but still resulted in a yield within an acceptable range for evaluation of the effects of enhancement.

Objective color scores (Table 17) were affected by days of display and enhancement (P<0.05), but not by PB (P>0.05) or any interactions (P>0.05). The L* values were greater on day 1 than on day 3 (P<0.05), but were not different from L* values on day 5 (P>0.05). Enhancement treatment did not affect (P>0.05) L* value. The a* values were greater on day 1 compared to day 3 and day 5 (P<0.0001; Table 17), indicating a decrease in redness of chops after the first day of storage. Also, a* values for E chops were less than those of N chops, (P=0.0007), showing that Enhanced chops were less red than Non-enhanced across all days of storage. The b* scores were also greater on day 1 compared to day 3 and day 5 (P=0.0125; Table 17), suggesting a decrease in yellowness of chops after the first day of storage. Additionally, Enhanced chops had lower b* values compared to Non-enhanced chops (P=0.0024), indicating that Enhanced chops were less yellow than Non-enhanced across all days of storage.

Results for TBARS testing conducted on day 1 and day 5 of storage are shown in Table 17. Similar to objective color scores, TBARS values were affected by days of storage (P<0.0001), but were not affected by PB treatment (P=0.1626) or any interactions (P>0.05). As expected, TBARS values (expressed in mg MDA per kg tissue) were increased on day 5 compared to day 1, indicating an increase in lipid oxidation with increased storage time. Enhancement with salt and phosphate tended (P=0.0997) to decrease TBARS values (Table 17). The mean TBARS values after 5 days of storage was 0.0973 mg MDA/kg tissue (Table 17), which is well below the threshold of detectable oxidative rancidity that has been generally reported (Tarladgis, Watts, Younathan, & Dugan, 1960; Wood et al., 2008). These low values may in some part be due to the very limited intramuscular fat in the goat loin chops, thereby limiting fat that was available for oxidation.

Consumer evaluation scores are shown in Table 18. Dietary PB and enhancement (ENH) treatment affected consumer sensory scores, but there were no interactions between PB and enhancement treatment (P>0.05). Appearance was not affected by PB (P=0.2556), but E had a greater score for appearance than N(P=0.0146). Likewise, aroma also was not affected by dietary PB (P=0.0776), but Enhanced had a greater score for aroma than Non-enhanced (P=0.0053), suggesting that consumers preferred the aroma of Enhanced loins over Non-enhanced loins. Consumer scores for texture were greater for 15 PB and 30 PB compared to OPB (P=0.0001; Table 18), indicating that consumers preferred the texture of 15 PB and 30 PB loins. Also, consumer scores for texture were greater (P<0.0001) for Enhanced loins (6.17, like slightly) than for Non-enhanced loins (5.05, neither like nor dislike). Flavor scores also improved with the addition of dietary PB. Consumer flavor scores were greater for the 15 PB and 30 PB loins compared to OPB (P=0.0321; Table 18), suggesting that consumers preferred the flavor of meat from goats fed dietary PB. Flavor scores were also improved by enhancement treatment treatment, such that consumer scores for flavor were greater (P<0.0001) for Enhanced loins (6.15, like slightly) than for Non-enhanced loins (5.31, neither like nor dislike). Overall acceptability scores were greater for the 15 PB and 30 PB treatments compared to OPB (P=0.0027; Table 18). Along with texture and flavor scores, consumers' overall acceptability ratings were greater (P<0.0001) for Enhanced loins (6.17, like slightly) than for Non-enhanced loins (5.34, neither like nor dislike). Enhancement with salt and phosphate is a widely used practice in pork and beef. Thus it is not surprising that consumer liking for all attributes was improved with ENH treatment of goat loin meat in the current study, and especially that texture, flavor, and overall acceptability scores were increased a full point as a result of enhancement treatment.

Consumer acceptance of goat meat seemed to be related to their previous experience eating chevon and their willingness to purchase a similar goat loin product. Of the consumers who completed the survey questions at the end of the consumer testing form, 39% had eaten goat meat before, and 61% had not. Also, 62% of consumers responded that they would be willing to purchase a goat meat product similar to the ones evaluated in the panel if it was available in the supermarket, while 38% responded that they would not be willing to buy a similar product. Consumers who had eaten goat meat before had a mean overall acceptability score of 6.11 (like slightly), while those who had not eaten goat meat prior to the panel had a mean overall acceptability score of 5.38 (neither like nor dislike) for all samples (Table 19). Moreover, the panelists who responded that they would be willing to purchase a goat meat product had a mean overall acceptability score of 6.28 (like slightly) for all samples, while those reported that they would not be willing to purchase goat meat had a mean overall acceptability score of 4.74 (dislike slightly) for all samples (Table 19).

Mean WBSF values are shown in Table 18. Dietary PB and enhancement treatment did not interact (P=0.2991) to affect WBSF. Mean WBSF values were greater for 0 PB loins than 30 PB loins (P=0.0199; Table 18). This suggests an improvement in tenderness with the addition of dietary PB to goat diets, and is reflected in the improved consumer texture scores for the 15 PB and 30 PB treatments. Enhancement treatment also decreased WBSF (P=0.0010; Table 18), showing that marination with salt and phosphate increased instrumental tenderness. Improvement in WBSF as a result of enhancement treatment was anticipated, as improvements in WBSF have been reported as a result of salt and phosphate enhancement in pork (Detienne et al., 2003) and lamb (Sawyer et al., 2003). Also, consumer liking of texture was greater for E loins compared to N loins in the current study, which reflects the decreased WBSF.

Results of the Experiment 4 indicate that the use of PB in goat diets had no impact on objective color or TBARS of goat loin meat after 5 days of storage. Dietary PB at 15 or 30% of the total diet led to a decrease in WBSF and an improvement in consumer evaluation of texture, flavor, and overall acceptability. Moreover, enhancement of loins with salt and phosphate led to an improvement in consumer evaluation of appearance, aroma, texture, flavor, and overall acceptability, as well as a decrease in WBSF. No interactions between PB and enhancement treatment affected any parameters measured in this study. These results suggest that PB may be successfully incorporated into livestock ruminant animal diets in geographical regions where it is economically advantageous to do so. Also, the use of enhancement techniques on meat obtained from such livestock does not appear to have differential effect on the meat when compared to livestock having a conventional diet. Thus, the methods of the present invention may be utilized with convention processing techniques to improve consumer acceptability and lead to expanded markets.

The various preferred embodiments and experiments having thus been described, those skilled in the art will readily appreciate that various modifications and variations can be made to the above described preferred embodiments without departing from the spirit and scope of the invention. The invention thus will only be limited to the claims as ultimately granted.

### Tables

TABLE 1
Ingredient and tannins composition of mixed
grain diet containing ground pine bark.

| Item | | | |
|---|---|---|---|
| Ingredient of the grain/pine | Control | 15% | 30% |
| bark mix, % as is | (%) | | |
| Ground pine bark | 0 | 15 | 30 |
| Ground wheat straw | 30 | 15 | 0 |
| Corn | 19 | 19 | 19 |
| Soy bean meal, 48% crude protein | 18.5 | 20 | 21 |
| Soy hulls | 4.5 | 5 | 4 |
| Alfalfa meal | 5 | 3 | 3 |
| Molasses | 6 | 6 | 6 |
| Vitamins and minerals | 0.5 | 0.5 | 0.5 |
| Burmudagrass hay | 15 | 15 | 15 |
| Condensed tannins, % DM | 0.19 | 1.63 | 3.2 |

TABLE 2
Effects of tannins-containing pine bark additive on animal
performance and feed efficiency traits in Boer-cross goat.

| | | | | | P-value | |
|---|---|---|---|---|---|---|
| | Treatment (% DMI) | | | | Lin- | Qua- |
| Item | 0 | 15 | 30 | SEM | ear | dratic |
| No. of | 8 | 7 | 7 | | | |
| animals | | | | | | |
| Initial BW | 27.39 | 27.53 | 27.34 | 1.04 | 0.97 | 0.91 |
| Final BW | 34.94 | 37.02 | 38.04 | 1.29 | 0.06 | 0.89 |
| ADG, g/d | 91.06 | 114.3 | 136.2 | 6.91 | 0.001 | 0.94 |
| Dry matter | | | | | | |
| Intake | | | | | | |
| (DMI), g/d | | | | | | |
| Grain | 1106.6 | 1142.9 | 1310.8 | 47.66 | 0.001 | 0.29 |
| mixture | | | | | | |
| Hay | 172.2 | 176.8 | 198.0 | 14.36 | 0.23 | 0.66 |
| Total DMI | 1278.8 | 1319.7 | 1508.8 | 54.02 | 0.001 | 0.303 |
| G:F ratio | 0.074 | 0.086 | 0.089 | 0.004 | 0.04 | 0.51 |

BW = body weight, ADG = average daily gain, DMI = dry matter intake, G:F ratio = gain:feed ratio.

TABLE 3
Effects of tannins-containing pine bark additive
on selected carcass characteristics of m. longissimus
muscle in Boer-Kiko cross-breed type.

| | | | | | P-value | |
|---|---|---|---|---|---|---|
| | Treatment (% DMI) | | | | Lin- | Qua- |
| Item | 0 | 15 | 30 | SEM | ear | dratic |
| No. of animals | 8 | 7 | 7 | | | |
| Empty BW, kg | 32.7 | 33.5 | 35.2 | 1.13 | 0.17 | 0.78 |
| HCW, kg | 15.9 | 16.5 | 16.8 | 0.65 | 0.40 | 0.85 |
| CCW, kg | 15.3 | 15.9 | 16.9 | 0.52 | 0.06 | 0.80 |
| Transportation shrink, % | 8.29 | 9.52 | 8.13 | 0.63 | 0.88 | 0.15 |
| Carcass Shrink, % | 3.86 | 3.64 | 3.85 | 0.29 | 0.97 | 0.60 |
| Dressing percentage | 48.7 | 49.5 | 48.0 | 1.31 | 0.63 | 0.47 |
| 12^{th} rib fat thickness, mm | 1.38 | 1.21 | 1.25 | 0.25 | 0.73 | 0.76 |
| REA, cm² | 8.22 | 8.34 | 8.14 | 0.30 | 0.87 | 0.72 |
| Body wall, mm | 12.9 | 11.8 | 11.8 | 0.94 | 0.21 | 0.49 |
| Leg circ, cm | 52.3 | 52.5 | 53.7 | 0.68 | 0.21 | 0.57 |
| Sirloin, kg | 1.18 | 1.21 | 1.36 | 0.04 | 0.01 | 0.29 |
| Loin, kg | 0.66 | 0.72 | 0.66 | 0.026 | 0.90 | 0.12 |
| Kidney fat, kg | 0.15 | 0.20 | 0.20 | 0.04 | 0.50 | 0.73 |
| Trim, kg | 0.91 | 0.91 | 1.11 | 0.04 | 0.01 | 0.15 |

CCW = cold carcass weight, HCW = hot carcass weight, REA = rib eye area, DP = dressing percentage = (HCW × 100)/fasting LW.

TABLE 4
Effects of ground pine bark additive on selected visceral organs in Boer-
Kiko cross-breed type goats.

| | Treatment (% DMI) | | | | P-value. | |
|---|---|---|---|---|---|---|
| Item | 0 | 15 | 30 | SEM | Linear | Quadratic |
| No. of animals | 8 | 7 | 7 | | | |
| Mass of organs, kg | | | | | | |
| Blood weight | 1.15 | 1.21 | 1.50 | 0.08 | 0.02 | 0.30 |
| Feet | 0.58 | 0.58 | 0.67 | 0.36 | 0.13 | 0.34 |
| Heart | 0.16 | 0.16 | 0.16 | 0.02 | 0.90 | 0.94 |
| Liver | 0.56 | 0.60 | 0.70 | 0.05 | 0.17 | 0.66 |
| Lungs | 0.50 | 0.50 | 0.62 | 0.03 | 0.04 | 0.18 |
| Hide | 5.03 | 4.93 | 6.11 | 0.21 | 0.01 | 0.05 |
| Gastrointestinal tract | 8.4 | 8.0 | 8.3 | 0.44 | 0.81 | 0.54 |
| (GIT), kg | | | | | | |
| Mass, % of empty BW | | | | | | |
| Blood weight | 3.4 | 3.6 | 3.8 | 0.17 | 0.19 | 0.95 |
| Feet | 1.7 | 1.7 | 1.8 | 0.05 | 0.21 | 0.96 |
| Heart | 0.44 | 0.47 | 0.43 | 0.02 | 0.63 | 0.27 |
| Liver | 1.7 | 1.8 | 2.0 | 0.06 | 0.02 | 0.53 |
| Lungs | 1.6 | 1.7 | 1.6 | 0.09 | 0.74 | 0.76 |
| Hide | 14.3 | 15.8 | 17.0 | 0.41 | 0.01 | 0.56 |
| Gastrointestinal tract | 25.8 | 23.7 | 23.5 | 0.81 | 0.08 | 0.38 |
| (GIT) | | | | | | |

TABLE 5
Effects of levels of ground pine bark additive on rumen fermentation
parameters in Boer-Kiko cross-breed type

| | Treatment (% DMI) | | | | P-value. | |
|---|---|---|---|---|---|---|
| Item | 0 | 15 | 30 | SEM | Linear | Quadratic |
| No. of animals | 8 | 7 | 7 | | | |
| Rumen ammonia, | | | | | | |
| mg/dL | | | | | | |
| Day 0 | 10.3 | 14.1 | 12.4 | 1.70 | 0.45 | 0.25 |
| Day 40 | 12.5 | 11.5 | 11.4 | 1.36 | 0.61 | 0.83 |
| Day 83 | 11.6 | 9.8 | 8.2 | 0.62 | 0.003 | 0.99 |
| Ruminal VFA, mM | | | | | | |
| Day 0 | | | | | | |
| Acetate | 33.6 | 32.8 | 33.6 | 2.74 | 0.99 | 0.82 |
| Propionate | 14.9 | 10.1 | 10.0 | 1.44 | 0.04 | 0.22 |
| Butyrate | 6.16 | 5.54 | 5.41 | 0.98 | 0.41 | 0.69 |
| A:P ratio | 2.45 | 3.29 | 3.69 | 0.36 | 0.05 | 0.66 |
| Day 40 | | | | | | |
| Acetate | 48.8 | 46.7 | 37.9 | 2.87 | 0.01 | 0.38 |
| Propionate | 10.7 | 12.6 | 14.1 | 0.84 | 0.01 | 0.87 |
| Butyrate | 5.0 | 6.5 | 10.5 | 1.10 | 0.03 | 0.42 |
| A:P ratio | 4.45 | 3.88 | 2.63 | 0.29 | 0.001 | 0.40 |
| Day 83 | | | | | | |
| Acetate | 35.4 | 30.8 | 28.6 | 2.67 | 0.07 | 0.70 |
| Propionate | 10.3 | 11.3 | 10.0 | 0.70 | 0.76 | 0.18 |
| Butyrate | 5.70 | 6.40 | 3.80 | 0.15 | 0.41 | 0.39 |
| A:P ratio | 3.45 | 2.73 | 2.85 | 0.10 | 0.01 | 0.01 |

VFA = volatile fatty acids,
A:P ratio = acetate:propionate ratios

TABLE 6
The influences of level of ground pine bark additive on the in vivo fecal dry
matter (DM) out-put and in vitro fecal methane gas production from goats fed grain
mixed ration.

| | | Treatment¹ | | | | | P-value². | |
|---|---|---|---|---|---|---|---|---|
| Parameter | Period | n | Control | 15% | 30% | SEM | Linear | Quadrate |
| Animal BW, kg | 1 | 6 | 32.3 | 32.5 | 32.9 | 1.38 | | |
| | 2 | 6 | 32.5 | 33.9 | 34.5 | 1.38 | | |
| | Mean | | 32.4 | 33.4 | 33.7 | 0.98 | 0.37 | 0.78 |
| Fecal DM out-put | 1 | 6 | 395.1 | 336.3 | 466.5 | 38.69 | | |
| (DM g/day) | 2 | 6 | 355.3 | 435.1 | 453.5 | 38.69 | | |
| | Mean | | 375.31 | 385.7 | 460.0 | 27.36 | 0.04 | 0.35 |
| Methane gas/g feces | 1 | 3 | 0.94 | 0.56 | 0.15 | 0.04 | | |
| | 2 | 3 | 0.59 | 0.29 | 0.08 | 0.04 | | |
| | Mean | | 0.77 | 0.42 | 0.11 | 0.03 | 0.001 | 0.65 |
| Estimated total fecal methane | 1 | 6 | 374.2 | 192.9 | 66.1 | 25.67 | | |
| gas production (cc)/day/goat | 2 | 6 | 208.5 | 123.9 | 35.2 | 25.67 | | |
| | Mean | | 291.3 | 158.4 | 50.6 | 18.15 | 0.001 | 0.58 |
| In vitro gas production | | | | | | | | |
| Total gas production | 1 | 3 | 83.3 | 63.7 | 32.2 | 4.88 | | |
| (cc) | 2 | 3 | 61.7 | 43.3 | 23.0 | 4.88 | | |
| | Mean | | 72.5 | 53.5 | 27.6 | 3.45 | 0.001 | 0.43 |
| Total in vitro methane gas production, cc | 1 | 3 | 7.5 | 4.5 | 1.2 | 0.39 | | |
| | 2 | 3 | 4.7 | 2.3 | 0.6 | 0.39 | | |
| | Mean | | 6.2 | 3.4 | 0.89 | 0.26 | 0.001 | 0.65 |

¹Animals were fed grain mix that contained 0, 15, and 30% ground pine bark with two different periods.
²Based on orthogonal contrasts for equally spaced treatments.
DW = body weight,
DM = dry matter.

TABLE 7
Growth rate of total fecal bacteria and generic fecal E. coli in growing
goats fed 3 levels of ground pine bark additive

| | | Treatment | | | | P-value. | |
|---|---|---|---|---|---|---|---|
| Item | time | Control | 15% | 30% | SEM | Linear | Quadratic |
| Number of goats | | 8 | 7 | 7 | | | |
| Fecal bacteria | | | | | | | |
| Total fecal bacteria | February, d 0 | 6.76 | 6.64 | 6.81 | 0.12 | 0.81 | 0.36 |
| | April, d 50 | 6.60 | 7.12 | 6.02 | 0.69 | 0.56 | 0.35 |
| | May, d 80 | 6.88 | 7.33 | 6.80 | 0.22 | 0.81 | 0.08 |
| Generic fecal E. coli | February, d 0 | 5.67 | 6.07 | 6.23 | 0.37 | 0.33 | 0.83 |
| | April, d 50 | 5.17 | 6.45 | 5.14 | 0.55 | 0.97 | 0.68 |
| | May, d 80 | 6.62 | 6.65 | 6.99 | 0.35 | 0.47 | 0.75 |
| Skin swap bacteria | | | | | | | |
| Total bacteria | | 1.92 | 1.37 | 1.51 | 0.18 | 0.15 | 0.17 |
| E. coli | | 0.91 | 0.61 | 1.14 | 0.31 | 0.53 | 0.28 |

D = day.

TABLE 8
Daily fecal egg count (FEC) and packed cell volumes in growing Kiko-cross
breed goats fed different levels of ground pine bark additive.

| | | Treatment | | | | P-value. | |
|---|---|---|---|---|---|---|---|
| Item | Time | Control | 15% | 30% | SEM | Linear | Quadratic |
| Number of goats | | 8 | 7 | 7 | | | |
| Fecal egg count | Day 0 | 987.5 | 628.6 | 714.3 | 171.1 | 0.28 | 8.32 |
| | Day 10 | 543.8 | 350.0 | 464.3 | 143.5 | 0.70 | 0.41 |
| | Day 52 | 1531.3 | 442.9 | 600.0 | 351.4 | 0.04 | 0.19 |
| | Day 65 | 1456.3 | 585.7 | 546.4 | 319.3 | 0.03 | 0.41 |
| | Mean | 1129.7 | 501.8 | 546.4 | 131.2 | 0.003 | 0.05 |
| Packed cell volume, mm | Day 0 | 12.6 | 11.9 | 12.8 | 0.49 | 0.80 | 0.22 |
| | Day 29 | 13.2 | 12.6 | 12.9 | 0.69 | 0.76 | 0.65 |
| | Day 72 | 13.1 | 13.1 | 13.1 | 0.65 | 0.77 | 0.94 |
| | Mean | 12.6 | 12.9 | 12.9 | 0.35 | 0.79 | 0. |

TABLE 9
Ingredient and chemical composition of experimental diets including different
grain mixes containing pine bark (PB) and wheat straw (WS), and bermudagrass hay
(BGH).

| | Grain Mixes (% PB) | | | | | | | |
|---|---|---|---|---|---|---|---|---|
| Item | 0 | 15 | 30 | SEM | P-value | PB | WS | BGH |

(%)

Ingredient of the grain/pine bark mix, % as is

| | | | | | | | | |
|---|---|---|---|---|---|---|---|---|
| Ground pine bark | 0 | 15 | 30 | — | — | — | — | — |
| Ground wheat | 30 | 15 | 0 | — | — | — | — | — |
| Corn | 20 | 20 | 20 | — | — | — | — | — |
| Soy bean meal, 48% CP | 18.5 | 20 | 21 | — | — | — | — | — |
| Soy hulls | 4.5 | 5 | 4 | — | — | — | — | — |
| Alfalfa meal | 5 | 3 | 3 | — | — | — | — | — |
| Molasses | 6 | 6 | 6 | — | — | — | — | — |
| Vitamins and mineral mix | 0.5 | 0.5 | 0.5 | — | — | — | — | — |
| Salt | 0.5 | 0.5 | 0.5 | — | — | — | — | — |
| NH4CL | 0.5 | 0.5 | 0.5 | — | — | — | — | — |
| Bermudagrass hay | 15 | 15 | 15 | — | — | — | — | — |

Chemical composition of the grain mix, % dry matter (n = 3)

| | | | | | | | | |
|---|---|---|---|---|---|---|---|---|
| Dry matter | 89.7 | 87.8 | 87.3 | 0.77 | 0.59 | 83.6 | 83.5 | 91.4 |
| Crude protein | 15.7 | 16.8 | 16.1 | 0.41 | 0.17 | 1.2 | 4.1 | 7.3 |
| Acid detergent fiber | 23.7 | 23.2 | 23.6 | 1.42 | 0.96 | 72.1 | 49.2 | 37.3 |
| Neutral detergent fiber | 35.0 | 31.8 | 27.5 | 1.77 | 0.06 | 78.6 | 79.0 | 69.2 |
| NFC^{a} | | | | | | | | |
| Lignin | 5.9 | 9.9 | 12.4 | 0.85 | 0.01 | 21.3 | 8.01 | 6.29 |
| Ether Extract | 2.3 | 2.6 | 2.5 | 0.25 | 0.71 | 1.65 | 0.42 | 1.51 |
| Total digestible nutrient | 66.6 | 64.1 | 64.4 | 1.75 | 0.58 | 36.7 | 52.0 | 56.3 |
| Net Energyₘ (Mcal/kg) | 0.31 | 0.30 | 0.30 | 0.01 | 0.24 | 0.10 | 0.21 | 0.54 |
| Net Energy_{g} (Mcal/kg) | 0.19 | 0.17 | 0.18 | 0.01 | 0.26 | 0.10 | 0.10 | 0.28 |
| Ca | 0.61 | 0.56 | 0.53 | 0.04 | 0.51 | 0.25 | 0.17 | 0.39 |
| P | 0.35 | 0.38 | 0.37 | 0.02 | 0.51 | 0.04 | 0.08 | 0.19 |
| Mg | 0.23 | 0.23 | 0.24 | 0.01 | 0.85 | 0.02 | 0.05 | 0.24 |
| K | 1.19 | 1.12 | 1.05 | 0.03 | 0.10 | 0.03 | 0.31 | 0.99 |
| S | 0.21 | 0.22 | 0.22 | 0.09 | 0.57 | 0.01 | 0.01 | 0.20 |
| Na | 0.10 | 0.10 | 0.08 | 0.08 | 0.14 | 0.08 | 0.04 | 0.01 |
| Cu, ppm | 34.7 | 25.3 | 19.7 | 8.01 | 0.17 | 1.0 | 5.0 | 3.0 |
| Mn, ppm | 118.3 | 108.3 | 94.3 | 12.0 | 0.42 | 30.0 | 63.0 | 43.0 |
| Zn, ppm | 133.0 | 142.3 | 152.0 | 14.6 | 0.67 | 11.0 | 5.0 | 20.0 |
| Fe, ppm | 192.7 | 203.6 | 196.6 | 19.09 | 0.91 | 384 | 111 | 211.3 |
| CT, % dry matter | 0.19 | 1.63 | 3.20 | 0.19 | 0.01 | 11.0 | 0.03 | — |

^{a}Condensed tannins (CT) are relative to a purified Quebracho condensed tannins standard (on dry matter basis).
^{b}Non fibrous carbohydrate

TABLE 10
Effects of condensed tannin-containing pine bark (PB) supplementation on
animal performance in Kiko-cross goat kids.

| | | | | | P-value^{a}. | |
|---|---|---|---|---|---|---|
| | Grain Mixes (% PB) | | | | | Quad- |
| Item | 0 | 15 | 30 | SEM | Linear | ratic |
| No. of | 8 | 7 | 7 | | | |
| animals | | | | | | |
| Initial | 27.4 | 27.5 | 27.3 | 1.04 | 0.97 | 0.91 |
| BW | | | | | | |
| Final | 34.9 | 37.0 | 38.0 | 1.29 | 0.06 | 0.89 |
| BW | | | | | | |
| ADG, | 91.1 | 114.3 | 136.2 | 6.91 | 0.001 | 0.94 |
| g/d | | | | | | |
| DMI/ | | | | | | |
| g/d | | | | | | |
| Grain | 1106.6 | 1142.9 | 1310.8 | 47.66 | 0.001 | 0.29 |
| mix | | | | | | |
| Hay | 172.2 | 176.8 | 198.0 | 14.36 | 0.23 | 0.66 |
| Total | 1278.8 | 1319.7 | 1508.8 | 54.02 | 0.001 | 0.30 |
| DMI | | | | | | |
| G:F | 0.074 | 0.086 | 0.089 | 0.004 | 0.04 | 0.51 |
| ratio | | | | | | |

^{a}Based on orthogonal contrast for equally spaced treatments.
BW = body weight,
ADG = average daily gain,
DMI = dry matter intake,
G:F = gain:feed ratios.

TABLE 11
Effects of condensed tannin-containing pine bark (PB) supplementation on
rumen fermentation parameters in Kiko-cross goat kids.

| | Grain Mixes (% PB) | | | | P-value^{a}. | |
|---|---|---|---|---|---|---|
| Item | 0 | 15 | 30 | SEM | Linear | Quadr. |
| No. of animals | 8 | 7 | 7 | | | |
| Rumen ammonia, mg/dL | | | | | | |
| D 0 | 10.3 | 14.1 | 12.4 | 1.70 | 0.45 | 0.25 |
| D 40 | 12.5 | 11.5 | 11.4 | 1.36 | 0.61 | 0.83 |
| D 83 | 11.6 | 9.8 | 8.2 | 0.62 | 0.003 | 0.99 |
| Ruminal VFA, mM | | | | | | |
| Day 0 | | | | | | |
| Acetate | 33.6 | 32.8 | 33.6 | 2.74 | 0.99 | 0.82 |
| Propionate | 14.9 | 10.1 | 10.0 | 1.44 | 0.04 | 0.22 |
| Butyrate | 6.16 | 5.54 | 5.41 | 0.98 | 0.41 | 0.69 |
| A:P ratio | 2.45 | 3.29 | 3.69 | 0.36 | 0.05 | 0.66 |
| Day 40 | | | | | | |
| Acetate | 48.8 | 46.7 | 37.9 | 2.87 | 0.01 | 0.38 |
| Propionate | 10.7 | 12.6 | 14.1 | 0.84 | 0.01 | 0.87 |
| Butyrate | 5.0 | 6.5 | 10.5 | 1.10 | 0.03 | 0.42 |
| A:P ratio | 4.45 | 3.88 | 2.63 | 0.29 | 0.001 | 0.40 |
| Day 83 | | | | | | |
| Acetate | 35.4 | 30.8 | 28.6 | 2.67 | 0.07 | 0.70 |
| Propionate | 10.3 | 11.3 | 10.0 | 0.70 | 0.76 | 0.18 |
| Butyrate | 5.7 | 6.4 | 3.8 | 1.5 | 0.41 | 0.39 |
| A:P ratio | 3.45 | 2.73 | 2.85 | 0.10 | 0.01 | 0.01 |

^{a}Based on orthogonal contrast for equally spaced treatments.
VFA = volatile fatty acids,
A:P = acetate: propionate ratios

TABLE 12
Effects of condensed tannin-containing pine bark (PB) supplementation on
selected carcass characteristics of LM in Kiko-cross goat kids.

| | Grain Mixes (% PB) | | | | P-value^{a}. | |
|---|---|---|---|---|---|---|
| Item | 0 | 15 | 30 | SEM | Linear | Quadr. |
| No. of animals | 8 | 7 | 7 | | | |
| Empty BW, kg | 32.7 | 33.5 | 35.2 | 1.13 | 0.17 | 0.78 |
| HCW, kg | 15.9 | 16.5 | 16.8 | 0.65 | 0.40 | 0.85 |
| CCW, kg | 15.3 | 15.9 | 16.9 | 0.52 | 0.06 | 0.80 |
| Transportation shrink, % | 8.29 | 9.52 | 8.13 | 0.63 | 0.88 | 0.15 |
| Carcass Shrink, % | 3.86 | 3.64 | 3.85 | 0.29 | 0.97 | 0.60 |
| Dressing percentage | 48.7 | 49.5 | 48.0 | 1.31 | 0.63 | 0.47 |
| 12^{th} rib fat thickness, mm | 1.38 | 1.21 | 1.25 | 0.25 | 0.73 | 0.76 |
| LM area, cm² | 8.22 | 8.34 | 8.14 | 0.30 | 0.87 | 0.72 |
| Body wall fat, mm | 12.9 | 11.8 | 11.8 | 0.94 | 0.21 | 0.49 |
| Leg circle, cm | 52.3 | 52.5 | 53.7 | 0.68 | 0.21 | 0.57 |
| Sirloin, kg | 1.18 | 1.21 | 1.36 | 0.04 | 0.01 | 0.29 |
| Loin, kg | 0.66 | 0.72 | 0.66 | 0.026 | 0.90 | 0.12 |
| KPF, kg | 0.15 | 0.20 | 0.20 | 0.04 | 0.50 | 0.73 |
| Shoulder, kg | 3.3 | 3.4 | 3.7 | 0.14 | 0.14 | 0.69 |
| Breast, kg | 0.50 | 0.51 | 0.63 | 0.02 | 0.01 | 0.15 |
| Trim, kg | 0.91 | 0.91 | 1.11 | 0.04 | 0.01 | 0.15 |

HCW = hot carcass,
CCW = cold carcass weight;
dressing percentage = (HCW × 100)/fasting body weight (BW);
KPF = kidney pelvic fat,
LM = longissimus muscle.
^{a}Based on orthogonal contrast for equally spaced treatments.

TABLE 13
Effects of condensed tannin-containing pine bark (PB) supplementation on
selected visceral organs in Kiko-cross goat kids.

| | Grain Mixes (% PB) | | | | P-value^{a}. | |
|---|---|---|---|---|---|---|
| Item | 0 | 15 | 30 | SEM | Linear | Quadr. |
| No. of animals | 8 | 7 | 7 | | | |
| Mass of organs, kg | | | | | | |
| Blood weight | 1.51 | 1.21 | 1.50 | 0.08 | 0.02 | 0.30 |
| Feet | 0.58 | 0.58 | 0.67 | 0.36 | 0.13 | 0.34 |
| Heart | 0.16 | 0.16 | 0.16 | 0.02 | 0.90 | 0.94 |
| Liver | 0.56 | 0.60 | 0.70 | 0.05 | 0.17 | 0.66 |
| Lungs | 0.50 | 0.50 | 0.62 | 0.03 | 0.04 | 0.18 |
| Hide | 5.03 | 4.93 | 6.11 | 0.21 | 0.01 | 0.05 |
| Gastrointestinal tract | 8.4 | 8.0 | 8.3 | 0.44 | 0.81 | 0.54 |
| (GIT) | | | | | | |
| Mass, % of empty | | | | | | |
| body weight | | | | | | |
| Blood weight | 3.4 | 3.6 | 3.8 | 0.17 | 0.19 | 0.95 |
| Feet | 1.7 | 1.7 | 1.8 | 0.05 | 0.21 | 0.96 |
| Heart | 0.44 | 0.47 | 0.43 | 0.02 | 0.63 | 0.27 |
| Liver | 1.7 | 1.8 | 2.0 | 0.06 | 0.02 | 0.53 |
| Lungs | 1.6 | 1.7 | 1.6 | 0.09 | 0.74 | 0.76 |
| Hide | 14.3 | 15.8 | 17.0 | 0.41 | 0.01 | 0.56 |
| Gastrointestinal tract | 25.8 | 23.7 | 23.5 | 0.81 | 0.08 | 0.38 |
| (GIT) | | | | | | |

^{a}Based on orthogonal contrast for equally spaced treatments.

TABLE 14
Effects of condensed tannin-containing pine bark (PB) supplementation
on LM color parameters of Kiko-cross goat kids.

| | Grain Mixes (% PB) | | | | P-value^{a}. | |
|---|---|---|---|---|---|---|
| Item | 0 | 15 | 30 | SEM | Linear | Quadratic |
| No. of animals | 8 | 7 | 7 | | | |
| Meat color parameters | | | | | | |
| L* value | 4.12 | 41.5 | 41.9 | 0.75 | 0.52 | 0.97 |
| a* value | 13.2 | 12.6 | 12.1 | 0.83 | 0.42 | 0.95 |
| b* value | 5.32 | 5.32 | 4.93 | 0.54 | 0.65 | 0.78 |

L* values are a measure of lightness (higher value indicates a lighter color);
a* values are a measure of redness (higher value indicates a redder color);
b* values are a measure of yellowness (higher values indicates a more yellow color).
^{a}Based on orthogonal contrast for equally spaced treatments.

TABLE 15
Effects of condensed tannin-containing pine bark (PB) supplementation on
hemogram of Kiko-cross goat kids.

| | Grain Mixes (% PB) | | | | P-value^{a}. | |
|---|---|---|---|---|---|---|
| Item | 0 | 15 | 30 | SEM | Linear | Quadratic |
| No. of animals | 8 | 7 | 7 | | | |
| Hematology | | | | | | |
| Hemoglobin, g/dL | 9.8 | 9.7 | 10.1 | 0.21 | 0.46 | 0.33 |
| Hematocrit, % | 16.8 | 16.0 | 16.8 | 0.56 | 0.91 | 0.29 |
| Mean corpuscular volume, fl | 22.2 | 22.0 | 22.2 | 0.14 | 0.89 | 0.45 |
| Mean corpuscular hemoglobin, g/dL | 13.2 | 13.6 | 13.5 | 0.39 | 0.54 | 0.67 |
| Mean corpuscular hemoglobin | 59.5 | 61.9 | 61.3 | 1.98 | 0.54 | 0.55 |
| concentration, % | | | | | | |
| Red cell distribution width, % | 33.7 | 34.4 | 34.4 | 0.66 | 0.45 | 0.68 |
| Mean platelet volume, fl | 13.0 | 12.6 | 12.9 | 0.36 | 0.81 | 0.35 |
| White blood cell, ×10³/uL | 10.3 | 10.8 | 8.7 | 0.63 | 0.10 | 0.13 |
| Red blood cell, 10⁶/μL) | 7.6 | 7.3 | 7.9 | 0.29 | 0.34 | 0.17 |
| White blood cell (Diff. - absolute | | | | | | |
| count/μL; % total) | | | | | | |
| Lymphocyte | 44.1 | 46.5 | 46.2 | 2.17 | 0.50 | 0.62 |
| Neutrophil | 53.3 | 56.6 | 54.1 | 4.05 | 0.92 | 0.61 |
| Monocyte | 6.3 | 3.1 | 2.7 | 2.7 | 0.37 | 0.69 |
| Eosinophil | 0.7 | 1.2 | 0.9 | 0.27 | 0.65 | 0.22 |
| Basophil | 2.6 | 1.3 | 0.8 | 0.49 | 0.02 | 0.51 |

^{a}Based on orthogonal contrast for equally spaced treatments.

TABLE 16
Effects of condensed tannin-containing pine bark (PB) supplementation on
blood serum chemistry in Kiko-cross goat kids.

| | Grain Mixes (% PB) | | | | P-value^{a}. | |
|---|---|---|---|---|---|---|
| Item | 0 | 15 | 30 | SEM | Linear | Quadratic |
| No. of animals | 8 | 7 | 7 | | | |
| Cholesterol (mg/dL) | 68.6 | 62.4 | 62.8 | 3.80 | 0.29 | 0.50 |
| Enzymes | | | | | | |
| Creatine Kinase (CK; IU/L) | 202.3 | 198.8 | 179.6 | 10.71 | 0.15 | 0.56 |
| Alanine Transaminase (ALT; U/L) | 26.1 | 22.2 | 20.9 | 1.34 | 0.01 | 0.46 |
| Amylase (U/L) | 79.5 | 55.5 | 51.7 | 11.5 | 0.09 | 0.49 |
| Alkaline Phosphatase (ALP; U/L) | 308.0 | 357.8 | 308.8 | 60.9 | 0.9 | 0.53 |
| Gamma glutyltranspepdidase (GGT; U/L) | 29.1 | 31.9 | 31.7 | 1.81 | 0.33 | 0.52 |
| Aspartate aminotransferase (AST; U/L) | 83.5 | 80.1 | 66.1 | 3.79 | 0.01 | 0.22 |
| Blood serum protein, g/dL | | | | | | |
| Total protein | 6.4 | 6.7 | 6.4 | 0.18 | 0.85 | 0.18 |
| Albumin (ALB) | 2.7 | 2.5 | 2.4 | 0.05 | 0.01 | 0.96 |
| Blood serum metabolites | | | | | | |
| Bilirubin (hematoidin; direct; mg/dL) | 0.2 | 0.2 | 0.1 | 0.04 | 0.25 | 0.22 |
| Bilirubin (total; mg/dL) | 0.18 | 0.23 | 0.16 | 0.02 | 0.62 | 0.05 |
| Glucose (g/dL) | 63.3 | 64.6 | 64.1 | 1.43 | 0.72 | 0.64 |
| Blood urea nitrogen (mg/dL) | 21.0 | 22.7 | 21.9 | 1.05 | 0.55 | 0.32 |
| Creatinine (mg/dL) | 0.67 | 0.69 | 0.67 | 0.03 | 0.96 | 0.53 |
| Triglyceride (Trig; mg/dL) | 25.7 | 26.4 | 25.9 | 1.69 | 0.93 | 0.79 |
| Blood serum minerals, mg/dL | | | | | | |
| Ca | 9.4 | 9.4 | 9.1 | 0.13 | 0.08 | 0.56 |
| P | 6.6 | 6.8 | 6.9 | 0.29 | 0.53 | 0.96 |
| Blood serum electrolytes, mmol/L | | | | | | |
| Na | 144.4 | 142.2 | 137.9 | 1.86 | 0.02 | 0.64 |
| K | 5.1 | 5.3 | 4.9 | 0.16 | 0.38 | 0.07 |
| Cl | 109.2 | 107.9 | 104.9 | 1.38 | 0.03 | 0.63 |
| CO₂-LC (mM/L) | 20.9 | 18.5 | 19.1 | 0.68 | 0.07 | 0.11 |

^{a}Based on orthogonal contrast for equally spaced treatments.

TABLE 17
Effect of days of display, enhancement treatment (ENH) and interactions with dietary pine bark (PB)
on objective color (L*, a*, and b*) and thiobarbituric acid reactive substances (TBARS¹)
of boneless goat loin meat

| | Days of Display | | | | | ENH² | | | | Day * ENH | Day * PB | PB |
|---|---|---|---|---|---|---|---|---|---|---|---|---|
| | d 1 | d 3 | d 5 | SEM | P-value | E | N | SEM | P-value | P-value | P-value | P-value |
| L* | 41.53^{a} | 39.36^{b} | 40.14^{ab} | 0.6537 | 0.0717 | 39.93 | 40.76 | 0.5343 | 0.2846 | 0.2429 | 0.8278 | 0.3363 |
| a* | 15.13^{a} | 13.56^{b} | 12.97^{b} | 0.2404 | <0.0001 | 13.35^{b} | 14.43^{a} | 0.196 | 0.0007 | 0.2347 | 0.6824 | 0.1121 |
| b* | 7.56^{a} | 6.91^{b} | 6.91^{b} | 0.162 | 0.0125 | 6.78^{b} | 7.47^{a} | 13.57 | 0.0024 | 0.1937 | 0.8621 | 0.7455 |
| TBARS | 0.0415^{b} | n/a | 0.0973^{a} | 0.0068 | <0.0001 | 0.0614 | 0.0775 | 0.0068 | 0.0997 | 0.8282 | 0.413 | 0.1626 |

¹TBARS values are expressed as mg malondialdehyde (MDA) per kg tissue
²ENH = Enhancement treatments: E = enhanced by vacuum tumbling with water, salt and phosphate; N = non-enhanced
^{a,b}Means in the same row lacking a common superscript are different (P < 0.05).

TABLE 18
Effect of dietary pine bark (PB) and enhancement treatment (ENH) on consumer evaluation
and Warner-Bratzler shear force (WBSF) of boneless goat loin meat

| | PB Treatment | | | | | ENH² | | | | ENH * PB |
|---|---|---|---|---|---|---|---|---|---|---|
| | 0 | 15 | 30 | SEM | P-value | E | N | SEM | P-value | P-value |
| Appearance¹ | 6.11 | 6.34 | 6.18 | 0.098 | 0.2556 | 6.35^{a} | 6.08^{b} | 0.801 | 0.0146 | 0.8745 |
| Aroma | 5.85 | 6.14 | 6.03 | 0.094 | 0.0776 | 6.16^{a} | 5.86^{b} | 0.077 | 0.0053 | 0.7820 |
| Texture | 5.21^{b} | 5.65^{a} | 5.96^{a} | 0.125 | 0.0001 | 6.17^{a} | 5.05^{b} | 0.103 | <0.0001 | 0.7142 |
| Flavor | 5.48^{b} | 5.87^{a} | 5.84^{a} | 0.118 | 0.0321 | 6.15^{a} | 5.31^{b} | 0.097 | <0.0001 | 0.8138 |
| Overall Acceptability | 5.45^{b} | 5.84^{a} | 5.97^{a} | 0.111 | 0.0027 | 6.17^{a} | 5.34^{b} | 0.091 | <0.0001 | 0.8750 |
| WBSF, N | 49.64^{a} | 44.03^{ab} | 37.35^{b} | 3.043 | 0.0199 | 37.52^{b} | 49.83^{a} | 2.433 | 0.0010 | 0.2991 |

¹Consumers (n = 111) evaluated samples using a 9-point hedonic scale, where 1 = dislike extremely and 9 = like extremely.
²ENH = Enhancement treatments: E = enhanced by vacuum tumbling with water, salt and phosphate; N = non-enhanced
^{a,b}Means in the same row lacking a common superscript are different (P < 0.05).

TABLE 19
Consumers' mean overall acceptability
scores¹ for enhanced and non-enhanced goat loin
meat based on previous consumption and willingness to buy

| Question | Response = Yes | Response = No |
|---|---|---|
| Would you consider purchasing | 6.28 | 4.74 |
| a goat meat product similar to | | |
| the ones tasted if it was available | | |
| in the supermarket? | | |
| Have you ever consumed chevon | 6.11 | 5.38 |
| (goat meat) before? | | |

¹Overall acceptability was evaluated using a 9-point hedonic scale, where 1 = dislike extremely and 9 = like extremely.

### CITED REFERENCES

The following list of prior art references provide background information that will readily understood by one skilled in the art, and may assist the same in practicing the invention without undue experimentation.
1. AOAC. 1998. Official Methods of Analysis, 16th ed. Assoc. Off. Anal. Chem., Gaithersburg, Md.
2. Barry, T. N., and W. C. McNabb. 1999. The implications of condensed tannins on the nutritive value of temperate forages fed to ruminants. Br. J. Nutr. 81:263-272.
3. Beauchmin, K. A., S. M. McGinn, T. F. Martinez, and T. A. McAllister. 2007. Use of condensed tannin extract from quebracho trees to reduce methane emissions from cattle. J. Anim. Sci. 85:1990-1996.
4. Black, J. L., M. Gill, D. E. Beever, J. K. M. Thornley, and J. D. Oldham. 1987. Simulation of the metabolism of absorbed energy yielding nutrients in young sheep. Efficiency and utilization of acetate. J. Nutr. 117:105-115.
5. Brooker, J. D., L. A. O'Donovan, I. Skene, K. Clarke, L. Blackall, and P. Muslera. 1994. Streptococcus caprinus sp. November, a tannin resistant ruminal bacterium from feral goats. Lett. Appl. Microbiol. 18:313-318.
6. Chaney, A. L., and E. P. Marbach. 1962. Modified reagents for determination of urea and ammonia. Clin. Chem. 8:130-132.
7. Foley, W. J., G. R. Lason, and C. McArthur. 1999. Role of plant secondary metabolites in the nutritional ecology of mammalian herbivores: how far have we come in 25 years. Pages 130-209 in 5^{th} Inter. Sym. Nutr. Herbivores. Am. Soc. Anim. Sci. Savoy, II.
8. Frutos, P., M. Raso, G. Hervas, A. R. Mantecon, V. Perez, and F. Giraldez. 2004. Is there any detrimental effect when a chestnut hydrolysable tannin extract is included in the diet of finishing lambs?. Anim. Res. 53:127-136.
9. Goetsch, A. L., and M. L. Galyean. 1983. Influence of feeding frequency on passage of fluid and particle markers in steers fed a concentrate diet. Can. J. Anim. Sci. 63:727-730.
10. Haslem, E. 1989. Plant Polyphenols-Vegetable Tannins. Cambridge Univ. Press, U.K.
11. Hoskin, S. O., T. N. Barry, P. R. Wilson, and P. D. Kemp. 1999. Growth and carcass production of young farmed deer grazing sulla (Hedysarum coronarium), chicory (Cichorium intybus), or perennial ryegrass (Lolium perenne)/white clover (Trifolium repens) pasture in New Zealand. N.Z. J. Agri. Res. 42:83-92.
12. Jones, W. T. and J. L. Mangan. 1977. Complexes of the condensed tannins of sainfoin (Onobrychis viciifolia scop.) with fraction 1 leaf protein and with submaxillary mucoprotein, and their reversal by polyethylene glycol and pH. J. Sci. Food Agric. 28:126-136.
13. Krueger, W. K., H. Gutierrez-Bañuelos, G. E. Carstens, B. R. Min, W. E. Pinchak, R. R. Gomez, R. C. Anderson, N. K. Krueger, and T. D. A. Forbes. 2009. Effects of dietary tannin source on performance, feed efficiency, ruminal fermentation, and carcass and non-carcass traits in steers fed a high-grain. Anim. Feed Sci. Technol. 159:1-9.
14. Kokalis-Burelle, N., and R. Rodriguez-Kabana. 1994. Changes in populations of soil microorganisms, nematodes, and enzyme activity associated with application of powdered pine bark. Plant and Soil 162: 169-175.
15. MacRae, J. C., and M. J. Ulyatt. 1974. Quantitative digestion of fresh herbage by sheep. II. The site of digestion of some nitrogenous constituents. J. Agric. Sci. 82:309-320.
16. Makkar, H. P. S. 2003. Effects and fate of tannins in ruminant animals, adaptation to tannins, and strategies to overcome detrimental effects of feeding tannin-rich feeds. Small Rum. Res. 49:241-256.
17. Makkar, H. P. S., M. Blummel, and K. Becker. 1995. In vitro effects of and interactions between animals and saponins and fate of tannins in the rumen. J. Sci. Food Agric. 69:481-493.
18. McArthur, C., C. T. Robbins, A. E. Hagerman, and T. A. Hanley. 1993. Diet selection by a ruminant generalist browser in relation to plant chemistry. Can. J. Zool. 71:2236-2243.
19. McSweeney, C. S., B. Palmer, D. M. McNeil, and D. O. Krauswe. 2001. Microbial interactions with tannins: nutritional consequences for ruminants. Anim. Feed Sci. Technol. 91:83-93.
20. Min, B. R., and S. P. Hart. 2003. Tannins for suppression of internal parasites. J. Anim. Sci. 81 (E. Suppl. 2):E102-E109.
21. Min, B. R., T. N. Barry, W. C. McNabb, and P. D. Kemp. 1998. Effect of condensed tannins on the production of wool and on its processing characteristics in sheep grazing Lotus corniculatus. Aus. J. Agric. Res. 49:597-605.
22. Min, B. R., W. C. McNabb, T. N. Barry, and J. S. Peters. 2000. Solubilization and degradation of ribulose-1,5-bisphosphate carboxylase/oxygenase (EC 4.1.1.39; Rubisco) protein from white clover (Trifolium repens) and Lotus corniculatus by rumen microorganisms and the effect of condensed tannins on these processes. J. Agric. Sci., Cambridge 134:305-317.
23. Min, B. R., G. T. Attwood, T. N. Barry, and W. C. McNabb. 2002. Lotus corniculatus condensed tannins decrease in vivo populations of proteolytic bacteria and affect nitrogen metabolism in the rumen of sheep. Can. J. Microbiol. 48:911-921.
24. Min B. R., T. N., Barry, G. T., Attwood, and W. C. McNabb. 2003. The effect of condensed tannins on the nutrition and health of ruminants fed fresh temperate forages: a review. Anim. Feed Sci. Technol. 106:3-19.
25. Mukkur, T. K. S., and R. E. Bradley. 1974. Determination of the absolute numbers of leukocyte cell types in chicken. Poult. Sci. 53:221-223.
26. Murdiati, T. B., C. S. McSweeney, and J. B. Lowry. 1992. Metabolism in sheep of gallic acid, tannic acid and hydrolysable tannin from Terminalia oblongata. Aust. J. Agric. Res. 43:1307-1319.
27. Natt, M. P., and C. A. Herrick. 1952. A new diluent for counting the erythrocytes and leukocytes of the chicken. Poult. Sci. 31:735-737.
28. Nelson, K. E., A. N. Pel, P. Schofield, and S. Zinder. 1995. Isolation and characterization of an anaerobic ruminal bacterium capable of degrading hydrolysable tannins. Appl. Environ. Microbiol. 61:3293-3298.
29. Nietfeld, J. C. 2010. Field necropsy techniques and proper specimen submission for investigation of merging infectious diseases of food animals. Vet. Clin. Food Anim. 26:1-13.
30. Niezen J. H., T. S., Waghorn, W. A. G. Charleston, and G. C. Waghorn. 1995. Growth and gastrointestinal nematode parasitism in lambs grazing either lucerne (Medicago sativa) or sulla (Hedysarum coronarium) which contains condensed tannins. J. Agric. Sci. 125:281-289.
31. NRC. 2007. Nutrient Requirement of Sheep, Goats, Cervids and Camelids. Academy Press. Washington, D.C.
32. Pfander, W. H., S. E. Grebing, G, Hajny, and W. Tyree. 1969. The value of wood-derived products in ruminant nutrition. Pages 298-314 in Advance in Chem., Am. Chem. Soc.
33. Priolo, A., G. C. Waghorn, M. Lanza, L. Biondi, and P. Pennisi. 2000. Polyethylene glycol as a means for reducing the impact of condensed tannins in carob pulp: Effect on lambs growth performance and meat quality. J. Anim. Sci. 778:810-816.
34. Puchala, R., B. R. Min, A. L. Goetsch, and T. Sahlu. 2005. The effect of a condensed tannin containing forage on methane emission by goats. J. Anim. Sci. 83:182-186.
35. Ramirez-Restrepo, C. A., T. N. Barry, W. E. Pomroy, L. Lopez-Villalobosa, W. C. McNabb, and P. D. Kemp. 2005. Use of Lotus corniculatus containing condensed tannins to increase summer lamb growth under commercial dryland farming conditions with minimal anthelmintic drench input. Anim. Feed Sci. Technol. 122:197-217.
36. Satter, L. D., and L. L. Slyter. 1974. Effect of ammonia concentration on rumen microbial protein production in vitro. Br. J. Nutr. 32:199-208.
37. Silanikove, N., and D. Tiomkin, 1992. Toxicity induced by poultry litter consumption: effect on parameters reflecting liver function in beef cows. Anim. Prod. 54:203-209.
38. Silanikove, N., N. Gilboa, A. Perevolotsky, and Z. Nitsan. 1996. Goats fed tannin-containing leaves do not exhibit toxic syndromes. Small Rum. Res. 21:195-201.
39. Solaiman, S., J. Thomas, Y. Dupre, B. R. Min, N. Gurung, and T. H. Terrill. 2010. Effect of feeding sericea lespedeza hay on growth performance, blood metabolites, and carcass characteristics of Kiko crossbred male kids. Small Rum. Res. 93:149-156.
40. Terrill, T. H., A. M. Rowan, G. B. Douglas, and T. N. Barry. 1992. Determination of extractable and bound condensed tannin concentrations in forage plants, protein concentrate meals and cereal grains. J. Sci. Food Agric. 58:321-329.
41. Turner, K. E., S. Wildeus, and J. R. Collins. 2005. Intake, performance, and blood parameters in young goats offered high forage diets of lespedeza or alfalfa hay. Small Rumin. Res. 59: 15-23.
42. USDA. 2001. Institutional meat purchased specifications for fresh goat. Series 11. USDA, MRP, AMF, Livestock and Seed Program, Washington, D.C. Meat Grading Certification Branch.
43. Van Soest, P. J., J. B. Robertson, and B. A. Lewis. 1991. Methods for dietary fiber, neutral detergent fiber, and non starch polysaccharides in relation to animal nutrition. J. Dairy Sci. 74:3583-3597.
44. Waghorn, G. C., and T. N. Barry. 1987. Pasture as a nutrient source. Pages 21-37 in Livestock Feeding on Pasture. A. M. Nicol, ed. N. Z. Soc. Anim. Prod. Occ. Publ. No. 10.
45. Wang, Y., G. B. Douglas, G. C. Waghorn, T. N. Barry, A. G. Foote, and R. W. Purchas. 1996. Effect of condensed tannins upon the performance of lambs grazing Lotus corniculatus and Lucerne (Medicago sativa). J. Agric. Sci. 126:87-98.
46. Warner, R. D., P. J. Walker, G. A. Edridge and J. C. Barnett. 1998. Effect of marketing procedures and live weight change prior to slaughter and beef carcass and meat quality. Anim. Prod. in Australia 22:165-168.
47. Warris, P. D. S. C. Kestin, S. C. Brown and L. J. Wilkins. 1984. The time required for recovery from mixing stress in young bulls and preventing DFD. Meat Sci. 10:53-68.
48. Webb, E. C., N. H. Casey, and L. Simela. 2005. Goat meat quality. Small Rum. Res. 60:153-166.
49. Ahn, J., Grun, I. U., & Fernando, L. N. (2006). Antioxidant properties of natural plant extracts containing polyphenolic compounds in cooked ground beef. Journal of Food Science, 67, 1364-1369.
50. Batten, G. J, (1987). Kiko: A new meat goat breed. In: proceedings fo the 4th Int. Conf. on Goats. March 8-13, Brasilia, Brazil. Vol 2, pp. 1330-1338.
51. Browning, Jr. R & Browing, L. L. (2009). Reproductive, growth, and fitness traits among Boer, Kiko, and Spanish meat goats semi-intensively managed in the southeastern U.S. Tropical and Subtropical Agroecosystems. 11: 109-113.
52. Child, R. D., Byington, E. K., & Hansen, H. H. (1985). Goats in the Mixed hardwoods of the southeastern U.S. In: F. H. Baker and R. K. Jones (Ed.) Mutispecies Grazing. Winrock International. Marrilton, Ark.
53. Detienne, N. A., Reynolds, A. E., &Wicker, L. (2001). Phosphate marination of pork loins at high and low injection pressures. Journal of Food Quality. 26: 1-14.
54. Dubeuf, J.-P., Morand-Fehr, P., Rubino, R. (2004). Situation, changes and future of goat industry around the world. Small Ruminant Research. 51: 165-173.
55. Foley, W. J., Iason, G. R., & McArthur, C. (1999). Role of plant secondary metabolism on the nutritional energy of mammalian herbivores; how far have we come in 35 years Pages 130-209 in Nutritional Ecology of Herbivores. H. J. G. Jung, and G. C. Fabey, Jr. ed Fed. Am. Soc. Anim. Sci, Savoy, II.
56. Glimp, H. A. (1995). Meat goat production and marketing. J. Anim. Sci. 73: 291-295.
57. Glimp, H. A., Ospina, A. E., & Yazman, J. (1986). Strategies for expanding goat meat production, processing, and marketing in the southeastern U.S. Winrock International, Marrilton, Ark. P 58.
58. Gunderson, R. and Ospina, E. (1986). The structure of Arkansas agriculture: A taxonomy. Winrock International, Morrilton, Ark.
59. Kim, J. O., Kim, M. N., & Ha, Y. L. 1993. Processing of Korean's black goat's meat to tremove goaty flavor. Food Biotechnology. (Korea) 2: 26-29.
60. Lee, J. H., Vanguru, M., Kannan, G., Moore, D. A., Terril, T. H., & Kouakou, B. (2009). Influence of dietary condensed tannins from sericea lespedeza on bacterial loads in gastrointestinal tracts of meat goats. Livestock Science. 126: 314-317.
61. Mercado, S. (1991). Goat meat and skin: Supply demands and opportunities in Mexico. In: T. H. The (Ed.) National Symp. On Goat Meat Production and Marketing. P 61. Tulsa, Okla.
62. Min, B. R. & Hart, S. P. (2003). Tannins for suppression of internal parasites. J. Anim. Sci. 81: 102-109.
63. Molan, A. L., Attwood, G. T., Min, B. R., & McNabb, W. C. (2001). The effect of condensed tannins from Lotus pendunculatus and Lotus corniculatus on the growth of proteolytic rumen bacteria in vitro and their possible mode of action. Canadian Journal of Microbiology, 47, 626-633.
64. Priolo, A. & Vasta, V. (2007). Effects of tannin-containing diets on small ruminant meat quality. Italian Journal of Animal Science, 6, 527-530.
65. Oman, J. S., Waldron, D. F., Griffin, D. B., & Savell, J. W. (1999). Effect fo Breed-Type and Feeding Regimen on Goat Carcass Traits. J. Anim. Sci. 77: 3215-3218.
66. Park, Y. W. (1990). Effects of Breed, sex, and tissues on concentrations of macrominerals in goat meat. J. Food Science. 55: 308-311.
67. Puchala, R., Min, B. R., Goetsch, A. L., & Sahlu, T. (2005). The effect of a condensed tannin-containing forage on methane emission by goats. Journal of Animal Science. 83: 182-186.
68. Rhee, K. S., Ziprin, Y. A., Bishop, C. E., & Waldron, D. F. (1997). Composition and Stability of Goat Meat Patties as Affected by Breed Type and Feeding Regimen. Journal of Food Science. 62: 949-953.
69. Rojas, M. C. & Brewer, M. S. (2007). Effect of natural antioxidants on oxidative stability of cooked, refrigerated beef and pork. Journal of Food Science, 72(4), S282-S288.
70. Sawyer, J. T., Brooks, J. C., Apple, J. K., & Fitch, G. Q. (2009). Effects of solution enhancement on palatability and shelf-life characteristics of lamb retail cuts. Journal of Muscle Foods. 20(3): 352-366.
71. Sheard, P. R. & Tali, A. (2004). Injection of salt, tripolyphosphate and bicarbonate marinade solutions to improve the yield and tenderness of cooked pork loin. Meat Science. 68: 305-311.
72. Shelton, M. (1990). Goat Production in the United States. In: R. C. Gray (Ed.) Proc. Int. Goat Production Symp. Florida A & M Univ., Tallahassee.
73. Tarladgis, B. G., Watts, B. M., Younathan, M. T., & Dugan, L. (1960). A distillation method for the quantitative determination of malonaldehyde in rancid foods. Journal of the American Oil Chemists' Society, 37, 44-48.
74. Wood, J. D., Enser, M., Fisher, A. V., Nute, G. R., Sheard, P. R., Richardson, R. I., Hughes, S. I., & Whittington, F. M. (2008). Fat deposition, fatty acid composition and meat quality: A review. Meat Science, 78: 343-358.

1. A domesticated ruminant feed comprising a tannin-containing raw bark product, wherein the tannin-containing wood product is non-toxic.

2. The feed of claim 1 wherein the tannin-containing raw bark product comprises tannin-containing pine bark.

3. The feed of claim 1 wherein the tannin-containing raw bark product comprises condensed tannins.

4. The feed of claim 2 wherein the tannin containing pine bark is present in the feed at a range of 15-30%.

5. The feed of claim 3 wherein the feed comprises condensed tannins in the range of 0.19% to 3.2%.

6. The feed of claim 2 wherein the tannin containing pine bark is present in the feed at a range of 5 to 30%.

7. The feed of claim 3 wherein the feed comprises condensed tannins in the 1.63% to 3.2%.

8. (canceled)

9. A method of decreasing internal parasites comprising feeding a domesticated ruminant the feed of claim 2.

10. The method of claim 9 wherein the internal parasites are selected from the group consisting of E. coli., Flavobacteriaceae, Acinetobacter, Acinetobacter-baumannii, moraxellaceae.

11. The method of claim 9 wherein fecal egg counts are reduced by at least 50%.

12. The method of claim 9 wherein resistant worms are eliminated.

13. A method of decreasing fecal methane gas emissions by decreasing methanogenesis in domesticated ruminants by feeding the feed of claim 2.

14. A method of increasing feed efficiency by altering ruminal fermentation in domesticated ruminants by feeding the feed of claim 2.

15. A method of reducing the amount of phosphorous released from feces of ruminant animals in domesticated ruminants by feeding the feed of claim 2.
